(19) United States
(12) Patent Application Publication
(10) Pub. No.: US 2025/0188161 A1
(43) Pub. Date: Jun. 12, 2025

(54) TGF-ß ANTIBODIES

(71) Applicant: UNIVERSITE CATHOLIQUE DE LOUVAIN, Louvain-la-Neuve (BE)

(72) Inventors: Lore MARIËN, Gent (BE); Gitte DE BOECK, Gent (BE); Sophie LUCAS, Duisburg (BE); Fanny LAMBERT, Gembloux (BE); Sebastian Paul VAN DER WONING, Gent (BE); Stéphanie LIENART, Wezembeek-Oppem (BE)

(73) Assignee: UNIVERSITE CATHOLIQUE DE LOUVAIN, Louvain-la-Neuve (BE)

(21) Appl. No.: 18/845,158

(22) PCT Filed: Mar. 9, 2023
(86) PCT No.: PCT/EP2023/056082

§ 371 Date: Sep. 9, 2024

(30) Foreign Application Priority Data
Mar. 9, 2022 (GB) 2203269.2

(51) Int. Cl. C07K 16/22; A61K 39/00; A61P 37/06

(52) U.S. Cl. CPC C07K 16/22; A61P 37/06; A61K 2039/505; C07K 2317/35; C07K 2317/54; C07K 2317/75; C07K 2317/92

(57) ABSTRACT

Antibodies capable of activating TGF-β1, or antigen binding fragments thereof, in particular antibodies that can bind to latent TGF-β1 and release the mature TGF-β1 cytokine. Additionally, pharmaceutical compositions including the antibodies, or antigen binding fragments thereof. Also, methods in which the antibodies, or antigen binding fragments thereof, or the pharmaceutical compositions are administered to a subject in need thereof.

### FIELD OF THE INVENTION

The present invention relates to antibodies and antigen binding fragments thereof that bind to latent TGF-β1 and activate TGF-β1. Since TGF-β1 is an important immunosuppressive cytokine, the antibodies and antigen binding fragments described herein are useful in methods of suppressing pathological immune responses, for example in the prevention and treatment of autoimmune and allo-immune diseases. The antibodies and antigen binding fragments described herein may also be useful in methods of preventing and treating neoplastic disease.

### BACKGROUND TO THE INVENTION

TGF-β is a pleiotropic cytokine that regulates a multitude of biological processes including cell growth, differentiation, tissue homeostasis, and extracellular matrix remodelling. It plays important and complex roles in regulating the function of the immune system (as reviewed in Travis and Sheppard, Annu. Rev. Immunol. 2014; 32:51-82 and also Sanjabi et al., Cold Spring Harb Perspect Biol. 2017; 9: a022236). TGF-β1 null mice die postnatally but suffer excessive inflammation highlighting the importance of this cytokine in regulation of the immune response (Shull et al., Nature, 1992; 359:693-99).

Three isoforms of TGF-β are found in mammals: TGF-β1; TGF-β2; and TGF-β3. The predominant isoform expressed in the immune system is TGF-β1. All TGF-β isoforms are initially synthesized as precursor proteins consisting of a short signal peptide (to direct the TGF-β to the endoplasmic reticulum), a large N-terminal region termed the latency associated peptide (LAP) and a shorter C-terminal fragment that is the mature TGF-β cytokine. TGF-β is secreted from cells as a homodimer (including three inter-chain disulphide bonds) and either prior to or after secretion, the LAP is separated from the mature cytokine via furin cleavage. The N-terminal LAP remains non-covalently associated with the mature cytokine to form the latent TGF-β complex. In some cases, latent TGF-β associates with a family of regulatory proteins known as the “latent TGF-β binding proteins” or “LTBPs”. The complex of latent TGF-β with one or more LTBPs is sometimes referred to as the “large latent complex”.

The crystal structure of latent TGF-β has been solved and this has shed light on the means by which TGF-β converts from its latent form into its active form (Shi et al., Nature 2011; 474:343-49). The crystal structure has revealed that in the latent form, the mature cytokine region is encircled by the LAP such that the sites on the mature cytokine capable of contacting the TGF-β receptor complex are obscured. The mature cytokine is held in place through multiple contacts with a flexible loop structure within the LAP termed the “latency lasso”. Release of the inhibition mediated by the LAP is required for TGF-β activation.

TGF-β is activated in vivo via a variety of mechanisms. An important mode of activation is via binding of integrins to a linear tripeptide motif, “RGD”, within the LAP region of TGF-β. This binding of the LAP to integrins exerts a deforming physical force on the tethered latent TGF-β complex leading to release of the mature cytokine. Integrins that are particularly important in the activation of latent TGF-β include αvβ6, typically expressed by epithelial cells, and αvβ8, typically expressed by dendritic cells. Additional mechanisms by which latent TGF-β is activated in vivo may include binding to thrombospondin-1 (TSP-1) or alternatively by enzymatic activation, for example proteases such as serine proteases and metalloproteases cleaving the LAP region.

Once activated, TGF-β mediates its effects via binding to the TGF-β receptor complex at the cell surface. This complex consists of two transmembrane type I TGF-β receptors (TGF-βRI) and two transmembrane type II TGF-β receptors (TGF-βRII). Both receptors have a cytoplasmic region with serine/threonine kinase activity and binding of TGF-β to the receptor complex leads to the phosphorylation of TGF-βR1 by TGF-βRII. Phosphorylation at the receptor level triggers an intracellular signalling cascade whereby R-Smad proteins (Smad-2 and Smad-3) are recruited to the receptor complex and are subsequently phosphorylated. Phosphorylated Smad-2 and Smad-3 complex with Smad-4, and this Smad complex translocates to the nucleus to activate or repress gene expression via binding to Smad-responsive regulatory regions in the promoter segments of TGF-β-regulated genes. TGF-β binding to the TGF-β receptor complex can also trigger signalling via other well-characterised intracellular pathways and mediators including the MAP kinase cascade, the PI3K pathway and the Rho GTPase family. In most cell types, including normal and pre-malignant or malignant epithelial cells, signals induced by TGF-β1 are cytostatic. TGF-β1 thus exerts tumor suppressive activities on epithelial cells in early stages of cancer.

As noted above, TGF-β1 is also an important cytokine in the regulation of the immune response. TGF-β1 is produced by a wide variety of cells within the immune system and has effects at the level of multiple cell targets, particularly T cells. In this regard, TGF-β1 plays an important role in regulating differentiation of T cells into effector and regulatory subsets.

In its role as a key immunosuppressive cytokine, TGF-β1 is a potent inhibitor of T helper 1 (Th1) and T helper 2 (Th2) effector T cell differentiation and also a potent inhibitor of CD8+ T cell proliferation. In contrast, TGF-β1 actively promotes the generation of regulatory T cells (Tregs), both natural or thymic Tregs produced in the thymus early in life and also induced or peripheral Tregs generated from naïve CD4+ T cells in the periphery. In the thymus, TGF-β1 protects the Tregs from apoptosis during the developmental selection process and in the periphery, TGF-β1 promotes the conversion of naïve CD4+ T cells to Tregs by upregulating expression of the transcription factor Foxp3.

Activation of TGF-β1 at the surface of Tregs (and also certain other cell types) is regulated by binding to a transmembrane protein called “GARP” (Glycoprotein A repetitions predominant protein). Indeed, studies have revealed that GARP is required for the surface expression of latent TGF-β on activated Tregs, platelets and endothelial cells, and plays an important role in regulating the bioavailability of active TGF-β in the vicinity of Tregs (Tran et al., Proc Natl Acad Sci USA. 2009; 106:13445-50; Wang et al., Mol Biol Cell. 2012; 23:1129-39; Vermeersch et al., PLoS One. 2017; 12 (3): e0173329).

Given the importance of TGF-β1 signalling in regulating aspects of the immune response, attempts have been made to generate antibodies that bind to TGF-β1 and regulate its biological activity. International patent applications WO2015/015003 and WO2016/125017 describe antibodies capable of binding to the complex of GARP and latent TGF-β1 formed at the surface of Treg cells. These antibodies inhibit TGF-β1 activation and thereby dampen the immunosuppressive activity of the Treg population. These antibodies are thus proposed for use as immunostimulatory antibodies in the treatment of conditions or diseases where it is desirable to upregulate the immune response, for example for the immunotherapy of cancer.

However, there is still an unmet need for the generation of antibodies that bind to and activate TGF-β1 (or promote TGF-β1 activation), thereby increasing the immunosuppressive activity of the Treg population. These antibodies, object of the present invention, are proposed for use as immunoinhinbitory antibodies in the treatment of conditions or diseases where it is desirable to downregulate the immune response, for example in the treatment of autoimmune and allo-immune diseases or conditions. They are also proposed for use in the prevention or treatment of conditions or diseases where it is desirable to increase the tumor-suppressive, cytostatic activity of TGF-β1, for example in pre-malignant, primary or secondary metastatic stages of cancer.

### SUMMARY OF THE INVENTION

The present invention provides antibodies capable of activating TGF-β1, i.e., antibodies that can bind to latent TGF-β1 and release the mature TGF-β1 cytokine from the inhibition exerted by the LAP region of the full-length TGF-β1 protein.

In a first aspect, the present invention provides an antibody, or an antigen binding fragment thereof, which binds to latent TGF-β1, wherein the antibody or antigen binding fragment thereof activates TGF-β1.

In certain embodiments, the antibodies or antigen binding fragments bind to an epitope comprising one or more amino acids from the region of human or murine latent TGF-β1 consisting of amino acids 87-222, wherein human latent TGF-β1 is represented by SEQ ID NO: 1 and murine latent TGF-β1 is represented by SEQ ID NO: 5.

In certain embodiments, the antibodies or antigen binding fragments bind to an epitope comprising one or more amino acids from the region of human or murine latent TGF-β1 consisting of amino acids 87-135, wherein human latent TGF-β1 is represented by SEQ ID NO: 1 and murine latent TGF-β1 is represented by SEQ ID NO: 5.

The antibody or antigen binding fragment of claim 1 or claim 2, which binds to an epitope comprising one or more amino acids from the region of human or murine latent TGF-β1 consisting of amino acids 136-200, wherein human latent TGF-β1 is represented by SEQ ID NO: 1 and murine latent TGF-β1 is represented by SEQ ID NO: 5.

In certain embodiments, the antibodies or antigen binding fragments bind to an epitope comprising one or more amino acids from the region of human or murine latent TGF-β1 consisting of amino acids 201-222, wherein human latent TGF-β1 is represented by SEQ ID NO: 1 and murine latent TGF-β1 is represented by SEQ ID NO: 5.

For embodiments wherein the antibody or antigen binding fragment binds to human latent TGF-β1, the antibody or antigen binding fragment may bind to an epitope comprising amino acid residues E119, K123, S138, R141, S209, R210, G212, E213, 1214, and/or E215 of human latent TGF-β1.

For embodiments wherein the antibody or antigen binding fragment binds to human latent TGF-β1, the antibody or antigen binding fragment may bind to an epitope comprising amino acid residues G212 and E215 of human latent TGF-β1.

For embodiments wherein the antibody or antigen binding fragment binds to murine latent TGF-β1, the antibody or antigen binding fragment may bind to an epitope comprising amino acid residues S138, R141, P145, E146, S175, L208, N209 and/or D212 of murine latent TGF-β1. For embodiments wherein the antibody or antigen binding fragment binds to murine latent TGF-β1, the antibody or antigen binding fragment may bind to an epitope comprising amino acid residues L208 and D212 of murine latent TGF-β1. For antibodies or antigen binding fragments that bind to murine latent TGF-β1 via an epitope comprising amino acid residues L208 and D212, the epitope may not comprise another amino acid residue from within the region of murine latent TGF-β1 consisting of amino acids 201-222.

In certain embodiments, the antibodies or antigen binding fragments activate TGF-β1 when latent TGF-β1 is complexed with GARP. In particular, the antibodies or antigen binding fragments may activate TGF-β1 when latent TGF-1 is bound to the surface of one or more cell types selected from: regulatory T cells (Tregs), megakaryocytes, platelets, B lymphocytes, endothelial cells, fibroblasts, mesenchymal cells and hepatic stellate cells. In preferred embodiments, the antibodies or antigen binding fragments activate TGF-31 when latent TGF-β1 is bound to the surface of regulatory T cells (Tregs).

In certain embodiments, the antibodies or antigen binding fragments activate TGF-β1 when latent TGF-β1 is associated with one or more cell types selected from: myeloid cells; lymphoid cells; hematopoietic cells; and cancer cells at a pre-malignant stage.

In certain embodiments, the antibodies or antigen binding fragments are bivalent, preferably IgG antibodies, more preferably IgG1 antibodies. In certain embodiments, the antibodies or antigen binding fragments comprise the CH1 domain, hinge region, CH2 domain and/or CH3 domain of a human IgG, preferably human IgG1.

In certain embodiments, the antigen binding fragment is selected from the group consisting of: an antibody light chain variable domain (VL); an antibody heavy chain variable domain (VH or VHH); a single chain antibody (scFv); a (scFv)2 fragment, a F(ab′)2 fragment; a Fab fragment; an Fd fragment; an Fv fragment; a one-armed (monovalent) antibody; diabodies; triabodies; tetrabodies; or any antigen binding molecule formed by combination, assembly or conjugation of such antigen binding fragments.

In certain embodiments, the antibodies or antigen binding fragments are defined with reference to the specific CDR, VH and/or VL sequences recited herein. For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions. The invention further provides antibodies or antigen binding fragments, which bind to the same epitope as the antibodies or antigen binding fragments defined herein with reference to specific SEQ ID NOs.

Also provided are isolated nucleic acids encoding the antibodies or antigen binding fragments, including polynucleotides encoding the VH, VHH and/or VL domains of the antibodies or antigen binding fragments described herein. The invention further provides an expression vector comprising the afore-mentioned nucleic acids operably linked to regulatory sequences, which permit expression of the antibody, antigen binding fragment, variable heavy chain domain (VH or VHH) or variable light chain domain (VL) in a host cell or cell-free expression system. Also provided are host cells or cell-free expression systems containing the afore-mentioned expression vectors.

The present invention also provides a pharmaceutical composition comprising an antibody or antigen binding fragment in accordance with the first or second aspect of the invention and at least one pharmaceutically acceptable carrier or excipient.

Further provided is an antibody or antigen binding fragment in accordance with the first or second aspect of the invention, or a pharmaceutical composition in accordance with the invention for use as a medicament.

In a further aspect, the present invention provides a method of preventing or treating an autoimmune disease and/or an alloimmune disease in a subject in need thereof, wherein the method comprises administering to the subject a therapeutically effective amount of an antibody or antigen binding fragment according to the preceding aspects of the invention, or a pharmaceutical composition in accordance with the invention. In certain embodiments, the disease or condition is selected from the group consisting of: Inflammatory Bowel Disease (IBD), multiple sclerosis (MS), graft-versus-host disease (GVHD), allograft rejection, antibody-mediated allograft rejection (AMR), allogenic islet graft rejection, alopecia areata, ankylosing spondylitis, antiphospholipid syndrome (APS), autoimmune Addison's disease, Alzheimer's disease, antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopeniarpura or idiopathic thrombocytopenia purpura or immune mediated thrombocytopenia), autoimmune urticaria, Behcet's disease, bullous pemphigoid (BP), cardiomyopathy, Castleman's syndrome, celiac spruce-dermatitis, chronic fatigue immune dysfunction syndrome, chronic inflammatory demyelinating polyneuropathy (CIDP), Churg-Strauss syndrome, cicatrical pemphigoid, CREST syndrome, cold agglutinin disease, COVID-19 mediated postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre syndrome (GBS), Goodpasture's syndrome, Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen plantus, lichen sclerosus, lupus erthematosis, systemic lupus erythematosus (SLE), lupus nephritis, membranous neuropathy, membranous nephropathy, Meniere's disease, mixed connective tissue disease, mucous membrane pemphigoid, graft type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Reynauld's phenomenon, Reiter's syndrome, rheumatoid arthritis (RA), sarcoidosis, scleroderma, Sjögren's syndrome, solid organ transplant rejection, stiff-man syndrome, takayasu arteritis, toxic epidermal necrolysis (TEN), Stevens Johnson syndrome (SJS), temporal arteristis/giant cell arteritis, thrombotic thrombocytopenia purpura, thyroid eye disease, ulcerative colitis, uveitis, warm autoimmune hemolytic anemia (wAIHA), dermatitis herpetiformis vasculitis, anti-neutrophil cytoplasmic antibody-associated vasculitides, vitiligo, and Wegner's granulomatosis.

In a further aspect, the present invention provides a method of preventing or treating a neoplastic disease in a subject in need thereof, wherein the method comprises administering to the subject a therapeutically effective amount of an antibody or antigen binding fragment according to the preceding aspects of the invention, or a pharmaceutical composition in accordance with the invention.

The invention also provides a kit comprising an antibody or antigen binding fragment in accordance with the first or second aspect of the invention, and optionally instructions for use.

### BRIEF DESCRIPTION OF THE DRAWINGS

FIG. 1. Fourteen monoclonal antibodies that activate latent TGF-β1.

A. Schematic representation of the results obtained by screening mAbs directed against GARP or latent TGF-β1 to search for TGF-β1-activating antibodies.

B. Representative results of TGF-β1-reporter assays in HEK-293 cells. HEK-293T cells were transfected to express human (top panel) or murine (bottom panel) GARP: TGF-β1 complexes, as well as Firefly luciferase under the control of a TGF-β1-responsive promoter (pCAGA-12), and Renilla luciferase under a constitutive promoter to control for transfection efficacy. Transfected cells were incubated with the indicated mAbs (20 μg/ml) or the indicated concentrations of recombinant human TGF-β1 (rhTGF-1). Bar graphs represent ratios of Firefly luciferase activity normalized to Renilla luciferase activity (mean of triplicate+SD). Hatched histograms represent antibodies that showed Firefly/Renilla ratios superior to that measured in the absence of rhTGF-β1 or mAb in at least 3 independent experiments. These mAbs were identified as TGF-β1-activating antibodies.

C. EC50 concentrations for the indicated TGF-β1-activating antibodies.

FIG. 2. Bivalent monospecific antibodies and requirement for GARP expression.

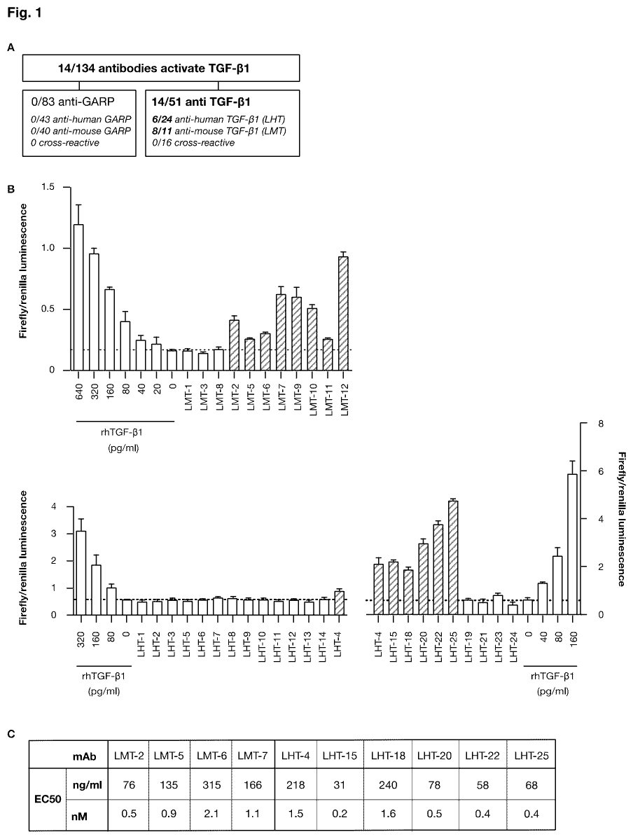

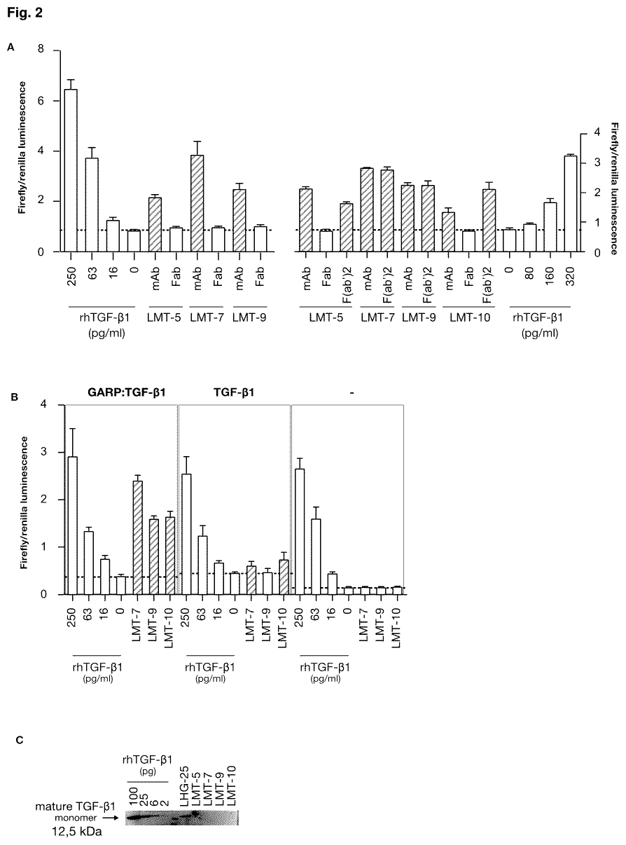

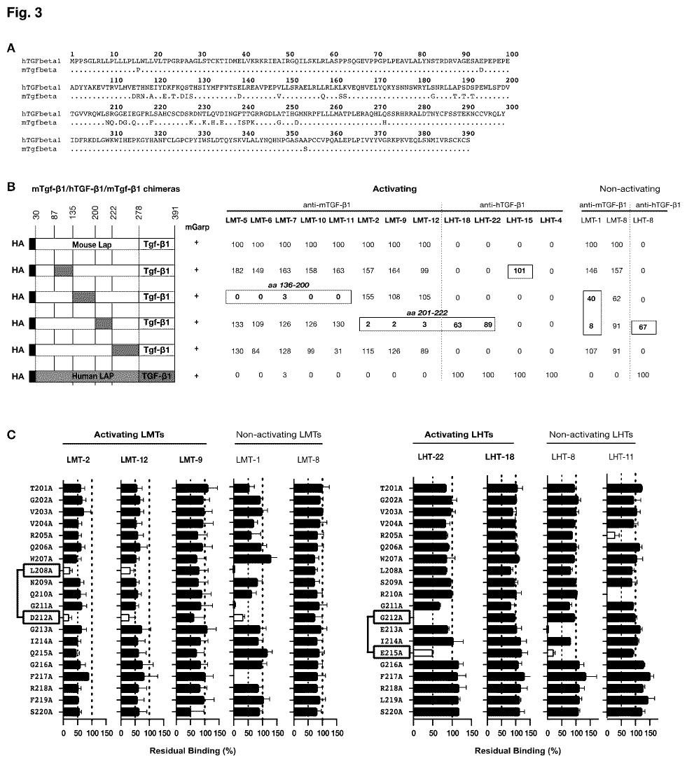

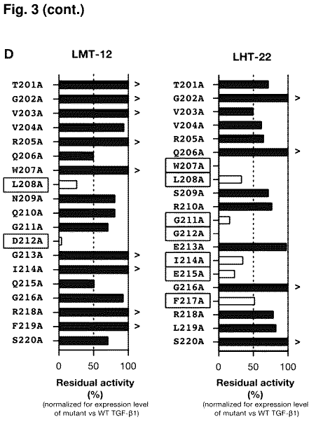

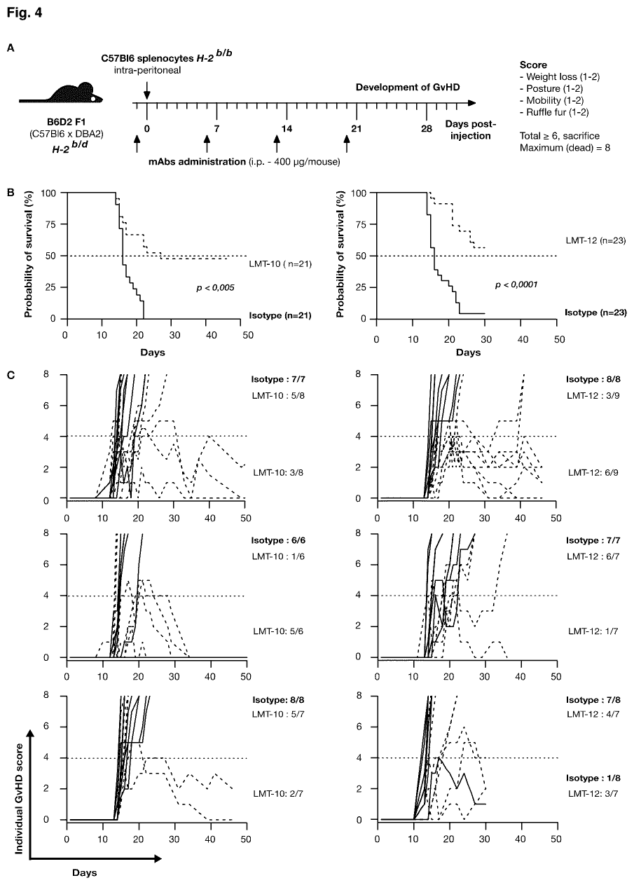

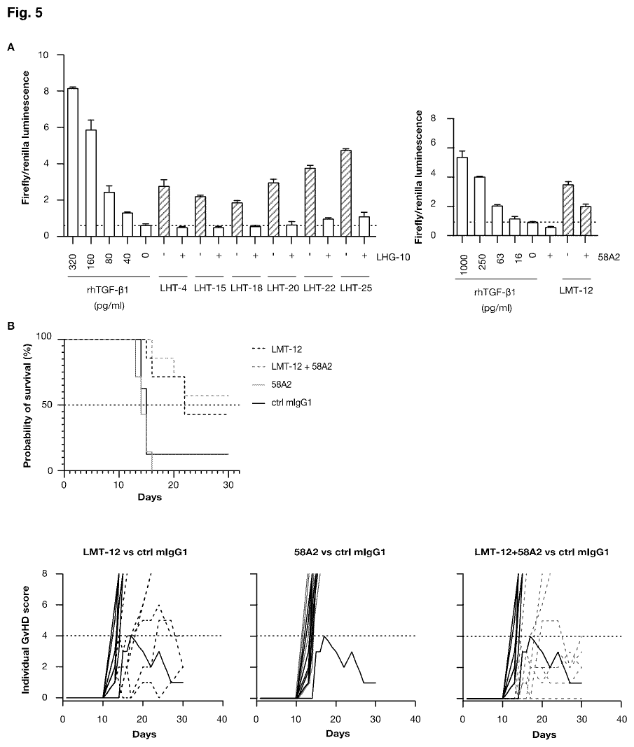

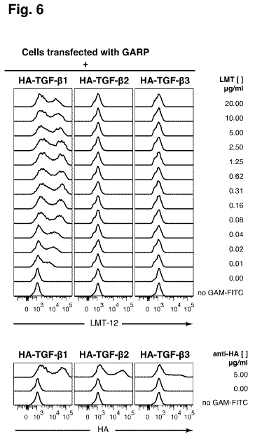

A. Antibodies activate mouse or human TGF-β1 when used as bivalent, monospecific full-length mAbs or Fab′2 fragments, but not as monovalent Fab fragments. HEK-293T cells were transfected as indicated in FIG. 1. Transfected cells were incubated with 20 μg/ml of the indicated full-length mAbs, Fab or Fab′2 fragments, or with the indicated concentrations of rhTGF-β1. Bar graphs represent ratios of Firefly luciferase activity normalized to Renilla luciferase activity (mean of triplicate+SD). Hatched histograms represent antibodies or antibody fragments that showed increased signals by comparison to incubation in absence of rhTGF-β1.

B. TGF-β1-activating antibodies activate latent TGF-β1 whether or not latent TGF-β1 is presented by GARP on the cell surface. Cells transfected and incubated with antibodies or rhTGF-β1 as indicated in A.

C. Western Blot analysis of TGF-β1 content in 25 μg of the indicated mAb preparation, or the indicated quantity of rhTGF-β1, using an anti-mature TGF-β1 antibody for ECL revelation.

FIG. 3. Epitopes bound by activating LMT and LHT mAbs are unique.

A. Sequence alignment of human and mouse TGF-β1 proteins. Signal peptide: amino-acids 1 to 29; LAP: amino-acids 30 to 278; mature TGF-β1: amino-acids 279 to 391.

B. Binding of LMT and LHT mAbs to HEK-293T cells transfected to express GARP: latent TGF-β1 complexes comprising the HA-tagged chimeric forms of TGF-β1 schematized on the left. Values on the right indicate % binding to a given chimera by comparison to binding to wild-type (WT) mouse TGF-β1 (100%).

C. Ala-scan analyses of antibody binding to Ala-mutants in region 201-222 of latent TGF-β1. Bar graphs indicate % binding to a given Ala-mutant by comparison to WT mouse TGF-β1 (100%). Binding below 50% are indicated by white bars. Mean of 2 to 3 experiments+SD.

D. Ala-scan analyses to identify residues in region 201-222 of latent TGF-β1 that are required for LHT-22 and LMT-12 to activate the cytokine. Bar graphs indicate % activation of the corresponding single-A mutant by comparison to WT TGF-β1 (normalized for expression level of mutant vs WT TGF-β1). Binding below 50% are indicated by white bars.

FIG. 4. LMT-10 and LMT-12 increased survival in a model of graft versus host disease.

A. Schematic representation of the experimental design. B6D2 F1 mice were injected i.p. with fresh splenocytes (70-90 millions) from C57BL/6 mice on day 0.400 μg of mAbs was injected i.p. once a week, starting on day-1. Mice were scored twice a week starting from day 0 and every day during the acute phase of the disease (days 13 to 20). Scores were determined based on weight loss (1 or 2 points), posture (1 or 2 points), mobility (1 or 2 points), ruffled fur (1 or 2 points). Mice were euthanized when they reached a score of 6.

B. Kaplan Meier plots representing the proportion of mice alive at the indicated days after transfer of splenocytes. P values calculated using a Gehan-Breslow Wilcoxon test (Graphpad Prism). Data pooled from three independent experiments.

C. Evolution of GvHD scores in individual mice measured in 3 independent experiments. Ratios indicated the proportion of mice dead from GvHD (above dashed line) or alive (below dashed line) 30-50 days after the transfer of allogeneic splenocytes.

FIG. 5. LMT-12 activated TGF-β1 from a GARP-independent source in a model of graft versus host disease.

A. Activation of latent TGF-β1 by activating mAbs is inhibited in the presence of a blocking anti-GARP mAb. HEK-293T cells were transfected and incubated with antibodies or the indicated concentration of rhTGF-β1, as indicated in FIG. 1. Bar graphs represent ratios of Firefly luciferase activity normalized to Renilla luciferase activity (mean of triplicate+SD). Hatched histograms represent conditions in which Firefly/Renilla ratios are superior to that measured in the absence of rhTGF-β1 or mAb

B. LMT-12 exerted the same therapeutic activity in GvHD whether it was administered alone or in combination with blocking anti-GARP-latent TGF-β1 antibody 58A2. Induction and scoring of GvHD was performed as described in FIG. 4. Top panel-Kaplan Meier plots representing the proportions of mice alive during the experiment, with P values calculated using a Gehan-Breslow Wilcoxon test (Graphpad Prism). Bottom panel-Evolution of GvHD scores in individual mice. Ratios indicated the proportion of mice dead from GvHD (above dashed line) or alive (below dashed line) 30-50 days after transplantation.

FIG. 6. Specificity testing of LMT-12 for mouse latent TGF-β1 The specificity of binding of mAb LMT-12 was tested by flow cytometry as described in Example 9. mAb LMT-12 was found to bind specifically to murine latent TGF-β1 but not murine latent TGF-β2 or murine latent TGF-β3.

### DETAILED DESCRIPTION

### A. Definitions

Unless defined otherwise, all technical and scientific terms used herein have the same meaning as is commonly understood by one skilled in the art in the technical field of the invention.

“Transforming growth factor β” or “TGF-β”—As used herein, the term “TGF-β” refers to the cytokine “Transforming growth factor β”. As described elsewhere herein, TGF-β is a pleiotropic cytokine that regulates a multitude of effects, particularly within the immune system. The term TGF-β as used herein is broad enough to cover the human isoforms of this protein and any species homologues. There are three isoforms of TGF-β in humans: TGF-β1, TGF-β2 and TGF-β3. The amino acid sequences of full-length human TGF-β1 and full-length murine TGF-β1 are shown in the table below as SEQ ID NOs: 1 and 5, respectively. These sequences correspond to the sequences deposited in the Uniprot database as: human TGF-β1-P01137-1; and mouse TGF-β1-P04202-1.

The full-length TGF-β1 protein can be divided up into: (i) the signal peptide (amino acid residues 1-29); (ii) the latency associated peptide or “LAP” (amino acid residues 30-278); and (iii) the mature cytokine region (amino acid residues 279-390). These three regions are shown below as SEQ ID NOs: 2, 3 and 4, respectively, for human TGF-β1 and as SEQ ID NOs: 6, 7 and 8, respectively, for murine TGF-β1.

TABLE 1
Human and Mouse TGF-β1 proteins

| | | SEQ ID NO. |
|---|---|---|
| Full-length | MPPSGLRLLPLLLPLLWLLVLTPGRPAAG | 1 |
| human TGF-β1 | LSTCKTIDMELVKRKRIEAIRGQILSKLR | |
| (390 aa) | LASPPSQGEVPPGPLPEAVLALYNSTRDR | |
| | VAGESAEPEPEPEADYYAKEVTRVLMVE | |
| | THNEIYDKFKQSTHSIYMFFNTSELREAV | |
| | PEPVLLSRAELRLLRLKLKVEQHVELYQK | |
| | YSNNSWRYLSNRLLAPSDSPEWLSFDVT | |
| | GVVRQWLSRGGEIEGFRLSAHCSCDSR | |
| | DNTLQVDINGFTTGRRGDLATIHGMNRP | |
| | FLLLMATPLERAQHLQSSRHRRALDTNY | |
| | CFSSTEKNCCVRQLYIDFRKDLGWKWIH | |
| | EPKGYHANFCLGPCPYIWSLDTQYSKVL | |
| | ALYNQHNPGASAAPCCVPQALEPLPIVY | |
| | YVGRKPKVEQLSNMIVRSCKCS | |
| Signal peptide for human | MPPSGLRLLPLLLPLLWLLVLTPGRPAAG | 2 |
| TGF-β1 (29 aa) | | |
| Latency associated peptide | LSTCKTIDMELVKRKRIEAIRGQILSKLR | 3 |
| for human TGF-β1 (249 aa) | LASPPSQGEVPPGPLPEAVLALYNSTRDR | |
| | VAGESAEPEPEPEADYYAKEVTRVLMVET | |
| | HNEIYDKFKQSTHSIYMFFNTSELREAVP | |
| | EPVLLSRAELRLLRLKLKVEQHVELYQK | |
| | YSNNSWRYLSNRLLAPSDSPEWLSFDVT | |
| | GVVRQWLSRGGEIEGFRLSAHCSCDSRD | |
| | NTLQVDINGFTTGRRGDLATIHGMNRP | |
| | FLLLMATPLERAQHLQSSRHRR | |
| Mature cytokine for human | ALDTNYCFSSTEKNCCVRQLYIDFRKDLG | 4 |
| TGF-β1 (112 aa) | WKWIHEPKGYHANFCLGPCPYIWSLDTQ | |
| | YSKVLALYNQHNPGASAAPCCVPQALEP | |
| | LPIVYYVGRKPKVEQLSNMIVRSCKCS | |
| Full-length murine TGF-β1 | MPPSGLRLLPLLLPLPWLLVLTPGRPAAG | 5 |
| (390 aa) | LSTCKTIDMELVKRKRIEAIRGQILSKLR | |
| | LASPPSQGEVPPGPLPEAVLALYNSTRDR | |
| | VAGESADPEPEPEADYYAKEVTRVLMVDR | |
| | NNAIYEKTKDISHSIYMFFNTSDIREAVP | |
| | EPPLLSRAELRLQRLKSSVEQHVELYQK | |
| | YSNNSWRYLGNRLLTPTDTPEWLSFDVT | |
| | GVVRQWLNQGDGIQGFRFSAHCSCDSKD | |
| | NKLHVEINGISPKRRGDLGTIHDMNRPF | |
| | LLLMATPLERAQHLHSSRHRRALDTNYC | |
| | FSSTEKNCCVRQLYIDFRKDLGWKWIHE | |
| | PKGYHANFCLGPCPYIWSLDTQYSKVLA | |
| | LYNQHNPGASASPCCVPQALEPLPIVYY | |
| | VGRKPKVEQLSNMIVRSCKCS | |
| Signal peptide for murine | MPPSGLRLLPLLLPLPWLLVLTPGRPAAG | 6 |
| TGF-β1 (29 aa) | | |
| Latency associated peptide | LSTCKTIDMELVKRKRIEAIRGQILSKLR | 7 |
| for murine TGF-β1 (249 aa) | LASPPSQGEVPPGPLPEAVLALYNSTRDR | |
| | VAGESADPEPEPEADYYAKEVTRVLMVDR | |
| | NNAIYEKTKDISHSIYMFFNTSDIREAVP | |
| | EPPLLSRAELRLQRLKSSVEQHVELYQK | |
| | YSNNSWRYLGNRLLTPTDTPEWLSFDVT | |
| | GVVRQWLNQGDGIQGFRFSAHCSCDSKD | |
| | NKLHVEINGISPKRRGDLGTIHDMNRPF | |
| | LLLMATPLERAQHLHSSRHRR | |
| Mature cytokine for murine | ALDTNYCFSSTEKNCCVRQLYIDFRKDLG | 8 |
| TGF-β1 (112 aa) | WKWIHEPKGYHANFCLGPCPYIWSLDTQ | |
| | YSKVLALYNQHNPGASASPCCVPQALEP | |
| | LPIVYYVGRKPKVEQLSNMIVRSCKCS | |

“GARP”—As used herein, the term “GARP” refers to the protein “Glycoprotein A repetitions predominant”, which is also known in the literature as “Leucine-rich repeat protein 32” or “LRRC32”. GARP is an 80 kDa type I transmembrane protein and serves as a receptor for latent TGF-β on the surface of inter alia regulatory T cells (Stockis et al., Eur J Immunol. 2009 December; 39 (12): 3315-22).

The amino acid sequence of full-length human GARP is shown below as SEQ ID NO: 67. This sequence corresponds to the sequence deposited in the Uniprot database as human GARP-Q14392-1.

(SEQ ID NO: 67)
MRPQILLLLALLTLGLAAQHQDKVPCKMVDKKVSCQVLGLLQVPSV
LPPDTETLDLSGNQLRSILASPLGFYTALRHLDLSTNEISFLQPG
AFQALTHLEHLSLAHNRLAMATALSAGGLGPLPRVTSLDLSGNSL
YSGLLERLLGEAPSLHTLSLAENSLTRLTRHTFRDMPALEQLDLH
SNVLMDIEDGAFEGLPRLTHLNLSRNSLTCISDFSLQQLRVLDLS
CNSIEAFQTASQPQAEFQLTWLDLRENKLLHFPDLAALPRLIYLN
LSNNLIRLPTGPPQDSKGIHAPSEGWSALPLSAPSGNASGRPLSQ
LLNLDLSYNEIELIPDSFLEHLTSLCFLNLSRNCLRTFEARRLGS
LPCLMLLDLSHNALETLELGARALGSLRTLLLQGNALRDLPPYTF
ANLASLQRLNLQGNRVSPCGGPDEPGPSGCVAFSGITSLRSLSLV
DNEIELLRAGAFLHTPLTELDLSSNPGLEVATGALGGLEASLEVL
ALQGNGLMVLQVDLPCFICLKRLNLAENRLSHLPAWTQAVSLEVL
DLRNNSFSLLPGSAMGGLETSLRRLYLQGNPLSCCGNGWLAAQLH
QGRVDVDATQDLICRFSSQEEVSLSHVRPEDCEKGGLKNINLIII
LTFILVSAILLTTLAACCCVRRQKFNQQYKA

“Antibody” or “Immunoglobulin”—As used herein, the term “immunoglobulin” includes a polypeptide having a combination of two heavy and two light chains whether or not it possesses any relevant specific immunoreactivity. “Antibodies” refer to such assemblies which have significant known specific immunoreactive activity to an antigen of interest (herein latent TGF-β1). The term “latent TGF-β1 antibodies” or alternatively, “TGF-β1 antibodies” is used herein to refer to antibodies, which exhibit immunological specificity for latent TGF-β1, including human latent TGF-β1, and if indicated, species homologues thereof. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are within the scope of the present invention. The following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (Y, u, a, 8, E) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region of an antibody allows the antibody to selectively recognize and specifically bind epitopes on antigens. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains.

The term “antibody” as used herein is also intended to encompass “VHH antibodies” or “Heavy-chain only antibodies”.

“VHH antibodies”—As used herein the term “VHH antibody” or “heavy chain-only antibody” refers to a type of antibody produced only by species of the Camelidae family, which includes camels, llama, alpaca. Heavy chain-only antibodies or VHH antibodies are composed of two heavy chains and are devoid of light chains. Each heavy chain has a variable domain at the N-terminus, and these variable domains are referred to as “VHH domains” in order to distinguish them from the variable domains of the heavy chains of the conventional heterotetrameric antibodies i.e. the VH domains, described above.

“Epitope”—As used herein, the term “epitope” means the region of the TGF-β1 protein to which the antibody binds. An antibody will typically bind to its respective TGF-β1 epitope via a complementary binding site on the antibody. The epitope to which the antibody binds will typically comprise multiple amino acids from the TGF-1 protein. The epitope may include amino acids that are contiguous in the TGF-β1 protein i.e. a linear epitope, or may include amino acids that are non-contiguous in the TGF-β1 protein i.e. a conformational epitope.

“Binding Site”—As used herein, the term “binding site” comprises a region of an antibody or antigen binding fragment which is responsible for selectively binding to the target antigen of interest (e.g. TGF-β1). Binding domains comprise at least one binding site. Exemplary binding domains include an antibody variable domain. The antibodies of the invention may comprise a single binding site or multiple (e.g., two, three or four) binding sites.

“Variable region” or “variable domain”—The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods, 2000; 20:267-279).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, MD., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, MD. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen binding site of antibodies. Structural analysis of antibodies revealed the relationship between the sequence and the shape of the binding site formed by the complementarity determining regions (Chothia et al., J. Mol. Biol. 227:799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

“CDR”—As used herein, the term “CDR” or “complementarity determining region” means the non-contiguous antigen binding sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 2
CDR definitions

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

“Framework region”—The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Constant region”—As used herein, the term “constant region” refers to the portion of the antibody molecule outside of the variable domains or variable regions. Immunoglobulin light chains have a single domain “constant region”, typically referred to as the “CL or CL1 domain”. This domain lies C terminal to the VL domain. Immunoglobulin heavy chains differ in their constant region depending on the class of immunoglobulin (γ, μ, α, δ, ε). Heavy chains γ, α and δ have a constant region consisting of three immunoglobulin domains (referred to as CH1, CH2 and CH3) with a flexible hinge region separating the CH1 and CH2 domains. Heavy chains μ and ε have a constant region consisting of four domains (CH1-CH4). The constant domains of the heavy chain are positioned C terminal to the VH domain.

The numbering of the amino acids in the heavy and light immunoglobulin chains run from the N-terminus at the forked ends of the Y configuration to the C-terminus at the bottom of each chain. Different numbering schemes are used to define the constant domains of the immunoglobulin heavy and light chains. In accordance with the EU numbering scheme, the heavy chain constant domains of an IgG molecule are identified as follows: CH1-amino acid residues 118-215; CH2-amino acid residues 231-340; CH3-amino acid residues 341-446. In accordance with the Kabat numbering scheme, the heavy chain constant domains of an IgG molecule are identified as follows: CH1-amino acid residues 114-223; CH2-amino acid residues 244-360; CH3-amino acid residues 361-477. The “Fc domain” or “Fc region” typically defines the portion of the constant region of a heavy chain including the CH2 and CH3 domains. The Fc region may also include some residues from the hinge region. The “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 3 below.

TABLE 3
Human hinge sequences

| IgG | Upper | Middle | Lower |
|---|---|---|---|
| | hinge | hinge | hinge |
| IgG1 | EPKSCDKTHT | CPPCP | APELLGGP |
| | (SEQ ID | (SEQ ID | (SEQ ID |
| | NO: 68) | NO: 69) | NO: 70) |
| IgG3 | ELKTPLG | CPRCP(EPKSC | APELLGGP |
| | DTTHT | DTPPPCPRCP)₃ | (SEQ ID |
| | (SEQ ID | (SEQ ID | NO: 73) |
| | NO: 71) | NO: 72) | |
| IgG4 | ESKYGPP | CPSCP | APEFLGGP |
| | (SEQ ID | (SEQ ID | (SEQ ID |
| | NO: 74) | NO: 75) | NO: 76) |
| IgG2 | ERK | CCVECPPPCP | APPVAGP |
| | (SEQ ID | (SEQ ID | (SEQ ID |
| | NO: 77) | NO: 78) | NO: 79) |

“Antigen binding fragment”—The term “antigen binding fragment” or “fragment”, as used herein, refers to a part or portion of an antibody or antibody chain comprising fewer amino acid residues than an intact or complete antibody or antibody chain. The term “antigen binding fragment” refers to a polypeptide fragment of an immunoglobulin or antibody that binds antigen or competes with intact antibody (i.e., with the intact antibody from which it was derived) for antigen binding (i.e., specific binding to TGF-β1). As used herein, the term “antigen binding fragment” includes, for example, an antibody light chain variable domain (VL), an antibody heavy chain variable domain (VH), a single chain antibody (scFv), a F(ab′)2 fragment, a Fab fragment, an Fd fragment, an Fv fragment, a one-armed (monovalent) antibody, diabodies, triabodies, tetrabodies or any antigen binding molecule formed by combination, assembly or conjugation of such antigen binding fragments. The term “antigen binding fragment” as used herein is further intended to encompass antibody fragments selected from the group consisting of unibodies, domain antibodies and nanobodies. Fragments can be obtained, e.g., via chemical or enzymatic treatment of an intact or complete antibody or antibody chain or by recombinant means.

“Fab”—A “Fab” or “Fab fragment” refers to a molecule composed of a heavy chain and light chain wherein the light chain consists of the VL domain and the one constant domain (CL, CK or CA) and the heavy chain consists of the VH domain and the CH1 domain only. A Fab fragment is typically one arm of a conventional Y-shaped immunoglobulin molecule. A Fab fragment can be generated from an immunoglobulin molecule by the action of the enzyme papain. Papain cleaves immunoglobulin molecules in the region of the hinge so as yield two Fab fragments and a separate Fc region.

“scFv” or “scFv fragment”—An “scFv” or “scFv fragment” means a single chain variable fragment. An scFv is a fusion protein of a VH domain and a VL domain of an antibody connected via a linker.

“Valency”—As used herein the term “valency” refers to the number of potential target or antigen binding sites in an antibody. Each target or antigen binding site specifically binds one antigen or a specific site on a target antigen. When an antibody comprises more than one target binding site, each target binding site may specifically bind the same or different molecules (e.g., may bind to different ligands or different antigens, or different epitopes on the same antigen).

“Specificity” and “Multispecific antibodies”—As used herein, the term “specificity” refers to the ability to bind (e.g., immunoreact with) a given target, e.g. TGF-β1. It is preferred that the antibodies described herein “specifically bind” to their target antigen, wherein the term “specifically bind” refers to the ability of any antibody to preferentially immunoreact with a given target. The antibodies described herein may be monospecific and contain one or more binding sites which specifically bind a particular target. The antibodies described herein may also adopt “multispecific antibody” formats, for example bispecific antibodies, wherein the multispecific antibody binds to two or more target antigens.

“Derived From”—As used herein the term “derived from” a designated protein (e.g. a camelid antibody or antigen binding fragment thereof) refers to the origin of the polypeptide or amino acid sequence. In one embodiment, the polypeptide or amino acid sequence which is derived from a particular starting polypeptide is a CDR sequence or sequence related thereto. In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence which is derived from a particular starting polypeptide or amino acid sequence has an amino acid sequence that is essentially identical to that of the starting sequence, or a portion thereof wherein the portion consists of at least 3-5 amino acids, at least 5-10 amino acids, at least 10-20 amino acids, at least 20-30 amino acids, or at least 30-50 amino acids, or which is otherwise identifiable to one of ordinary skill in the art as having its origin in the starting sequence. In one embodiment, the one or more CDR sequences derived from the starting antibody are altered to produce variant CDR sequences, e.g. affinity variants, wherein the variant CDR sequences maintain target antigen binding activity.

“Camelid-Derived”—In certain embodiments, the antibodies of the invention comprise framework amino acid sequences and/or CDR amino acid sequences derived from a camelid conventional antibody or a VHH antibody raised by active immunisation of a camelid. However, antibodies of the invention comprising camelid-derived amino acid sequences may be engineered to comprise framework and/or constant region sequences derived from a human amino acid sequence (i.e. a human antibody) or other non-camelid mammalian species. For example, a human or non-human primate framework region, heavy chain portion, constant region and/or hinge portion may be included in the TGF-β1 antibodies. In one embodiment, one or more non-camelid amino acids may be present in the framework region of a “camelid-derived” antibody, e.g., a camelid framework amino acid sequence may comprise one or more amino acid mutations in which the corresponding human or non-human primate amino acid residue is present. Moreover, camelid-derived VH and VL domains, or humanised variants thereof, may be linked to the constant domains of human antibodies to produce a chimeric molecule, as described elsewhere herein.

“Conservative amino acid substitution”—A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.

“Heavy chain portion”—As used herein, the term “heavy chain portion” includes amino acid sequences derived from the constant domains of an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. In one embodiment, an antibody or antigen binding fragment of the invention may comprise the Fc portion of an immunoglobulin heavy chain (e.g., a hinge portion, a CH2 domain, and a CH3 domain). In another embodiment, an antibody or antigen binding fragment of the invention may lack at least a portion of a constant domain (e.g., all or part of a CH2 domain). In certain embodiments, at least one, and preferably all, of the constant domains are derived from a human immunoglobulin heavy chain. For example, in one preferred embodiment, the heavy chain portion comprises a fully human hinge domain. In other preferred embodiments, the heavy chain portion comprises a fully human Fc portion (e.g., hinge, CH2 and CH3 domain sequences from a human immunoglobulin).

In certain embodiments, the constituent constant domains of the heavy chain portion are from different immunoglobulin molecules. For example, a heavy chain portion of a polypeptide may comprise a CH2 domain derived from an IgG1 molecule and a hinge region derived from an IgG3 or IgG4 molecule. In other embodiments, the constant domains are chimeric domains comprising portions of different immunoglobulin molecules. For example, a hinge may comprise a first portion from an IgG1 molecule and a second portion from an IgG3 or IgG4 molecule. As set forth above, it will be understood by one of ordinary skill in the art that the constant domains of the heavy chain portion may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant region domain (CL). Exemplary modifications include additions, deletions or substitutions of one or more amino acids in one or more domains.

“Chimeric”—A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric antibodies of the invention include fusion proteins comprising camelid-derived VH and VL domains, or humanised variants thereof, fused to the constant domains of a human antibody, e.g. human IgG1, IgG2, IgG3 or IgG4.

“Synthetic”—As used herein, the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature.

“Engineered”—As used herein, the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life or effector function.

“Modified antibody”—As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. scFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain portion lacking a CH2 domain and comprising a binding domain of a polypeptide comprising the binding portion of one member of a receptor ligand pair.

The term “modified antibody” may also be used herein to refer to amino acid sequence variants of the antibodies of the invention as structurally defined herein. It will be understood by one of ordinary skill in the art that an antibody may be modified to produce a variant antibody which varies in amino acid sequence in comparison to the antibody from which it was derived. For example, nucleotide or amino acid substitutions leading to conservative substitutions or changes at “non-essential” amino acid residues may be made (e.g., in CDR and/or framework residues). Amino acid substitutions can include replacement of one or more amino acids with a naturally occurring or non-natural amino acid.

“Humanising substitutions”—As used herein, the term “humanising substitutions” refers to amino acid substitutions in which the amino acid residue present at a particular position in the VH or VL domain of an antibody (for example a camelid-derived TGF-β1 antibody) is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of the antibodies, defined herein.

“Humanised variants”—As used herein the term “humanised variant” refers to a variant antibody which contains one or more “humanising substitutions” compared to a reference antibody, wherein a portion of the reference antibody (e.g. the VH domain and/or the VL domain or parts thereof containing at least one CDR) has an amino acid derived from a non-human species, and the “humanising substitutions” occur within the amino acid sequence derived from a non-human species.

“Germlined variants”—The term “germlined variant” is used herein to refer specifically to “humanised variants” in which the “humanising substitutions” result in replacement of one or more amino acid residues present at a particular position(s) in the VH or VL domain of a non-human antibody with an amino acid residue which occurs at an equivalent position in a reference human VH or VL domain encoded by the human germline. It is typical that for any given “germlined variant”, the replacement amino acid residues substituted into the germlined variant are taken exclusively, or predominantly, from a single human germline-encoded VH or VL domain. The terms “humanised variant” and “germlined variant” are often used interchangeably herein. Introduction of one or more “humanising substitutions” into a camelid-derived (e.g. llama derived) VH or VL domain results in production of a “humanised variant” of the camelid (llama)-derived VH or VL domain. If the amino acid residues substituted in are derived predominantly or exclusively from a single human germline-encoded VH or VL domain sequence, then the result may be a “human germlined variant” of the camelid (llama)-derived VH or VL domain.

“Affinity variants”—As used herein, the term “affinity variant” refers to a variant antibody which exhibits one or more changes in amino acid sequence compared to a reference antibody, wherein the affinity variant exhibits an altered affinity for the target antigen in comparison to the reference antibody. For example, affinity variants will exhibit a changed affinity for TGF-β1, as compared to the reference TGF-β antibody. Preferably the affinity variant will exhibit improved affinity for the target antigen as compared to the reference antibody. Affinity variants typically exhibit one or more changes in amino acid sequence in the CDRs, as compared to the reference antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

“High human homology”—An antibody comprising a heavy chain variable domain (VH) and a light chain variable domain (VL) may be considered as having high human homology if the VH domains and the VL domains, taken together, exhibit at least 90% amino acid sequence identity to the closest matching human germline VH and VL sequences. Antibodies having high human homology may include antibodies comprising VH and VL domains of native non-human antibodies which exhibit sufficiently high % sequence identity to human germline sequences, including for example antibodies comprising VH and VL domains of camelid conventional antibodies, as well as engineered, especially humanised or germlined, variants of such antibodies and also “fully human” antibodies.

In one embodiment the VH domain of the antibody with high human homology may exhibit an amino acid sequence identity or sequence homology of 80% or greater with one or more human VH domains across the framework regions FR1, FR2, FR3 and FR4. In other embodiments the amino acid sequence identity or sequence homology between the VH domain of the polypeptide of the invention and the closest matching human germline VH domain sequence may be 85% or greater, 90% or greater, 95% or greater, 97% or greater, or up to 99% or even 100%. VH domain variants having high human homology may have 1, 2, 3, 4, 5, 6, 7, 8, 9 or 10 amino acid substitutions relative to the original non-human VH domain sequence from which they derive.

In one embodiment the VH domain of the antibody with high human homology may contain one or more (e.g. 1 to 10) amino acid sequence mis-matches across the framework regions FR1, FR2, FR3 and FR4, in comparison to the closest matched human VH sequence.

In another embodiment the VL domain of the antibody with high human homology may exhibit a sequence identity or sequence homology of 80% or greater with one or more human VL domains across the framework regions FR1, FR2, FR3 and FR4. In other embodiments the amino acid sequence identity or sequence homology between the VL domain of the polypeptide of the invention and the closest matching human germline VL domain sequence may be 85% or greater 90% or greater, 95% or greater, 97% or greater, or up to 99% or even 100%. VL domain variants having high human homology may have 1, 2, 3, 4, 5, 6, 7, 8, 9 or 10 amino acid substitutions relative to the original non-human VL domain sequence from which they derive.

In one embodiment the VL domain of the antibody with high human homology may contain one or more (e.g. 1 to 10) amino acid sequence mis-matches across the framework regions FR1, FR2, FR3 and FR4, in comparison to the closest matched human VL sequence.

### B. TGF-31 Activating Antibodies that Bind Latent TGFβ-31

### Activation of TGF-31

In a first aspect, the present invention provides an antibody, or an antigen binding fragment thereof, which binds to latent TGF-β1, wherein the antibody or antigen binding fragment thereof activates TGF-β1. The antibodies and antigen binding fragments described herein may thus be referred to as any of the following: “activating TGF-β1 antibodies”; “TGF-β1 activating antibodies”; “anti-latent TGF-β1 activating antibodies”; or “anti-latent TGF-β1 antibodies activating the release of TGF-β1”.

As explained elsewhere herein, latent TGF-1 consists of a TGF-β1 homodimer wherein the two LAP regions of the TGF-β proteins have been cleaved from the mature TGF-β1 cytokine regions at the C-termini. The two N-terminal LAP polypeptides remain covalently associated with the C-terminal mature TGF-β1 cytokine thereby preventing TGF-β1 from binding to its receptor complex and initiating downstream signalling cascades. The antibodies and antigen binding fragments described herein are capable of binding to the latent TGF-β1 complex and releasing the mature TGF-β1 cytokine from the inhibition mediated by the LAP regions. This may involve physical dissociation of the LAP polypeptides from the mature TGF-β1 cytokine. Alternatively, the antibodies or antigen binding fragments described herein may bind to latent TGF-β1 resulting in deformation of the latent complex such that the mature TGF-β1 cytokine is exposed and can bind to the TGF-β receptor complex. The form of mature TGF-β1 that is free of inhibition from the LAP and is capable of binding to the TGF-β receptor complex is referred to herein as “active TGF-β1”.

As used herein, “activation of TGF-β1” means the conversion of latent TGF-β1 into active TGF-β1. This activation may be measured by a variety of methods. Such methods include but are not limited to in vitro methods such as Western blotting, ELISA, TGF-β1 reporter assays, and TGF-β1 activity assays.

In certain embodiments, TGF-β1 activation is measured by exposing cells expressing the TGF-β receptor complex to a combination of latent TGF-β1 and an antibody or antigen binding fragment described herein and detecting an increase in the phosphorylation status of the TGF-β receptor complex. Alternatively or in addition, TGF-β1 activation may be measured by detecting an increase in phosphorylation of the R-Smad proteins, e.g. Smad-2. The phosphorylation status of the TGF-β receptor complex and/or the R-Smad proteins (e.g. Smad-2) may be measured using any suitable technique including but not limited to Western blotting and ELISA. The level of phosphorylation observed when cells are exposed to the combination of latent TGF-β1 and an antibody or antigen binding fragment of the invention may be compared to one or more controls. For example, cells expressing the TGF-β receptor complex may be exposed to a range of concentrations of active TGF-β1 such that the phosphorylation status of the TGF-β receptor complex and/or R-Smad proteins (e.g. Smad-2) can be determined in the presence of known concentrations of active TGF-β1. The level of phosphorylation seen when antibodies or antigen binding fragments of the invention bind to latent TGF-β1 can thus be compared with the activation seen in the presence of standard concentrations of active TGF-β1.

In certain embodiments, TGF-β1 activation may be measured using in vitro assays wherein changes in gene expression serve as the read-out of TGF-β1 activation. Such assays include but are not limited to TGF-β1 reporter assays such as the reporter assay described in the Examples presented herein below. TGF-1 reporter assays typically involve use of a TGF-β1-responsive reporter construct wherein a TGF-β1 responsive element (as found for example in the promoter region of TGF-β1 responsive genes) is linked to a reporter gene such that activation of TGF-β1 produces an increase in the expression of the reporter gene. The TGF-β1 responsive element may be a Smad complex binding element, for example a nucleotide sequence comprising or consisting of (CAGA)₁₂ (as described in Dennler et al., EMBO J. 1998; 17:3091-3100).

The reporter gene may be any gene encoding a protein, the expression of which can be measured and/or quantitated. Preferred reporter genes include but are not limited to reporter genes encoding GFP and luciferase. The TGF-β1 reporter constructs exemplified herein incorporate a luciferase reporter gene such that an increase in luminescence can be used as a measure of TGF-β1 activation.

The complete TGF-β1 reporter assay may comprise the steps of: (i) engineering cells to carry a TGF-β1 reporter construct as described herein; (ii) exposing said cells to a combination of latent TGF-β1 and one or more TGF-β1 activating antibodies or antigen binding fragments; (iii) measuring the expression of the reporter gene. As exemplified herein, the cells engineered to carry the TGF-β1 reporter may also be engineered so as to express the latent TGF-β1 protein. In certain embodiments, the cells to be used in the TGF-β1 reporter assay are engineered so as to express both latent TGF-β1 and GARP such that the latent TGF-β1 is tethered at the cell surface. Controls may be incorporated into the assay, such as described herein, to control for transfection efficiency with respect to the TGF-β1 reporter construct.

As described above for assays based upon measuring phosphorylation levels, the level of activity seen in TGF-β1 reporter assays may be compared with one or more controls. For example, cells carrying a TGF-β1 reporter construct may be exposed to one or more concentrations of active TGF-β1 such that the read-out from the TGF-β1 reporter construct can be standardised for one or more known concentrations of active TGF-β1. The activation of the TGF-β1 reporter construct seen when antibodies or antigen binding fragments of the invention bind to latent TGF-β1 can thus be compared with the activation seen in the presence of standard concentrations of active TGF-β1.

Alternatively or in addition to the above, the activation mediated by the antibodies and antigen binding fragments described herein may be measured relative to the activation mediated under standard or physiological conditions found in vitro or in vivo. As described elsewhere herein, latent TGF-β1 can be activated via a variety of mechanisms including binding of the “RGD” motif within the LAP to integrins, binding to thrombospondin and by cleavage via proteases. Given that latent TGF-β1 binding to αVβ6 and αVβ8 integrins is well-established as a physiological mechanism by which latent TGF-β1 is activated in vivo, activation in the presence of the antibodies or antigen binding fragments described herein may be measured in vitro relative to the activation mediated by αVβ6 and/or αVβ8 integrins. A level of activation equivalent to or greater than the activation seen with integrins may classify the antibodies as “TGF-β1 activating antibodies”.

The antibodies or antigen binding fragments of the invention may activate TGF-β1 by binding to latent TGF-β1 in uncomplexed form i.e. when latent TGF-β1 is not bound by regulatory proteins such as LTBPs or GARP. Alternatively or in addition, the antibodies or antigen binding fragments of the invention may activate TGF-31 by binding to latent TGF-β1 when latent TGF-β1 is complexed with one or more additional proteins. In certain embodiments, the antibodies or antigen binding fragments of the invention activate TGF-β1 when latent TGF-β1 is complexed with GARP. As described elsewhere herein, GARP is a transmembrane protein expressed by certain cell types including regulatory T cells (Tregs), B lymphocytes, megakaryocytes, platelets, endothelial cells and fibroblasts. Antibodies or antigen binding fragments capable of activating TGF-β1 by binding to latent TGF-β1 complexed with GARP may lead to localised activation of TGF-β1 within the body i.e. in the vicinity of cell types expressing GARP. In certain preferred embodiments, the antibodies or antigen binding fragments described herein activate TGF-β1 when latent TGF-β1 is bound to the surface of Tregs. In certain embodiments, the antibodies or antigen binding fragments described herein activate TGF-β1 when latent TGF-β1 is bound to the surface of megakaryocytes, platelets and/or endothelial cells. In certain embodiments, the antibodies or antigen binding fragments described herein activate TGF-1 when latent TGF-β1 is bound to the surface of B lymphocytes, fibroblasts, mesenchymal cells and/or hepatic stellate cells.

Latent TGF-β1 may be associated with or tethered to the surface of cells via proteins other than GARP, for example by binding to LTBPs or other non-peptidic molecules. In certain embodiments, the antibodies or antigen binding fragments described herein activate TGF-β1 associated with or tethered to the surface of one or more cell types selected from the group consisting of: myeloid cells, lymphoid cells, hematopoietic cells, and cancer cells at a pre-malignant stage.

The activating TGF-β1 antibodies or antigen binding fragments of the invention may bind to human latent TGF-β1 and/or to murine latent TGF-β1. The sequence of human latent TGF-β1 is represented by SEQ ID NO: 1 and the sequence of murine latent TGF-β1 is represented by SEQ ID NO: 5. The activating TGF-31 antibodies and antigen binding fragments described herein typically bind either human latent TGF-β1 or murine latent TGF-1 i.e. they are not cross-reactive between these two species. The activating TGF-β1 antibodies or antigen binding fragments of the invention may exhibit binding specificity for TGF-1, i.e. the antibodies or antigen binding fragments bind to TGF-β1 but do not bind to other isoforms, specifically TGF-β2 and TGF-β3.

### Epitopes

The activating TGF-β1 antibodies or antigen binding fragments of the invention may be characterised by their unique binding epitopes within latent TGF-β1.

In some embodiments, the binding of the amino acids of the TGF-β1 antibodies or antigen binding fragments to the epitope within latent TGF-β1 refers to physical interactions well known in the field, comprising covalent bound or hydrogen bound.

In some embodiments, the interfacing of the amino acids of the TGF-31 antibodies or antigen binding fragments to the epitope within latent TGF-β1 refers to nonphysical interactions contributing to the activation of latent TGF-β1.

In certain embodiments, the TGF-β1 antibodies or antigen binding fragments bind to an epitope comprising one or more amino acids from the region of TGF-β1 consisting of amino acids 87-222 (wherein these amino acid positions are defined with respect to the full-length sequence of the human and mouse TGF-β1 proteins as represented by SEQ ID NOs: 1 and 5, respectively). In certain embodiments, the epitope comprises two amino acids from within this region.

In certain embodiments, the TGF-β1 antibodies or antigen binding fragments bind to an epitope comprising one or more amino acids from the region of TGF-β1 consisting of amino acids 87-135 (wherein these amino acid positions are defined with respect to the full-length sequence of the human and mouse TGF-β1 proteins as represented by SEQ ID NOs: 1 and 5, respectively). In certain embodiments, the epitope comprises two amino acids from within this region.

In certain embodiments, the TGF-β1 antibodies or antigen binding fragments bind to an epitope comprising one or more amino acids from the region of TGF-β1 consisting of amino acids 136-200 (wherein these amino acid positions are defined with respect to the full-length sequence of the human and mouse TGF-β1 proteins as represented by SEQ ID NOs: 1 and 5, respectively). In certain embodiments, the epitope comprises two amino acids from within this region.

In certain embodiments, the TGF-β1 antibodies or antigen binding fragments bind to an epitope comprising one or more amino acids from the region of TGF-β1 consisting of amino acids 201-222 (wherein these amino acid positions are defined with respect to the full-length sequence of the human and mouse TGF-β1 proteins as represented by SEQ ID NOs: 1 and 5, respectively). In certain embodiments, the epitope comprises two amino acids from within this region.

In certain embodiments, the TGF-1 antibodies or antigen binding fragments bind to a conformational epitope requiring one or more amino acids from the region of TGF-β1 consisting of amino acids 87-222 (wherein these amino acid positions are defined with respect to the full-length sequence of the human and mouse TGF-β1 proteins as represented by SEQ ID NOs: 1 and 5, respectively). In certain embodiments, the conformational epitope requires two amino acids from within this region.

In certain embodiments, the TGF-β1 antibodies or antigen binding fragments bind to a conformational epitope requiring one or more amino acids from the region of TGF-β1 consisting of amino acids 87-135 (wherein these amino acid positions are defined with respect to the full-length sequence of the human and mouse TGF-β1 proteins as represented by SEQ ID NOs: 1 and 5, respectively). In certain embodiments, the conformational epitope requires two amino acids from within this region.

In certain embodiments, the TGF-β1 antibodies or antigen binding fragments bind to a conformational epitope requiring one or more amino acids from the region of TGF-β1 consisting of amino acids 136-200 (wherein these amino acid positions are defined with respect to the full-length sequence of the human and mouse TGF-β1 proteins as represented by SEQ ID NOs: 1 and 5, respectively). In certain embodiments, the conformational epitope requires two amino acids from within this region.

In certain embodiments, the TGF-β1 antibodies or antigen binding fragments bind to a conformational epitope requiring one or more amino acids from the region of TGF-β1 consisting of amino acids 201-222 (wherein these amino acid positions are defined with respect to the full-length sequence of the human and mouse TGF-β1 proteins as represented by SEQ ID NOs: 1 and 5, respectively). In certain embodiments, the conformational epitope requires two amino acids from within this region.

The region of TGF-β1 consisting of amino acids 87-222 differs between the human and mouse TGF-β1 proteins as shown below:

Human latent TGF-β1: (aa 87- 222)
(SEQ ID NO: 82)
RVAGESAEPEPEPEADYYAKEVTRVLMVETHNEIYDKFKQSTHSI
YMFFNTSELREAVPEPVLLSRAELRLLRLKLKVEQHVELYQKYSN
NSWRYLSNRLLAPSDSPEWLSFDVTGVVRQWLSRGGEIEGFRLSA
H
Murine latent TGF-β1: (aa 87-222)
(SEQ ID NO: 83)
RVAGESADPEPEPEADYYAKEVTRVLMVDRNNAIYEKTKDISHSI
YMFFNTSDLREAVPEPVLLSRAELRLQRLKSSVEQHVELYQKYSN
NSWRYLGNRLLTPTDTPEWLSFDVTGVVRQWLNQGDGIQGFRFSA
H

The region of TGF-β1 consisting of amino acids 87-135 differs between the human and mouse TGF-β1 proteins as shown below:

Human latent TGF-β1: (aa 87-135)
(SEQ ID NO: 84)
RVAGESAEPEPEPEADYYAKEVTRVLMVETHNEIYDKFKQSTHSIYMFF
Murine latent TGF-β1: (aa 87-135)
(SEQ ID NO: 85)
RVAGESADPEPEPEADYYAKEVTRVLMVDRNNAIYEKTKDISHSIYMFF

The region of TGF-β1 consisting of amino acids 136-200 differs between the human and mouse TGF-β1 proteins as shown below:

Human latent TGF-β1: (aa 136-200)
(SEQ ID NO: 86)
NTSELREAVPEPVLLSRAELRLLRLKLKVEQHVELYQKYSNNSWR
YLSNRLLAPSDSPEWLSFDV
Murine latent TGF-β1: (aa 136-200)
(SEQ ID NO: 87)
NTSDLREAVPEPVLLSRAELRLQRLKSSVEQHVELYQKYSNNSWR
YLGNRLLTPTDTPEWLSFDV

The region of TGF-β1 consisting of amino acids 201-222 is located within the LAP region; more specifically, it corresponds to amino acids 172-193 of the LAP region (as represented by SEQ ID NOs: 3 and 7-human and murine, respectively). This region is located close to the dimerization interface of the latent TGF-β1 protein (see Shi et al., Nature 2011; 474:343-49). The “latency loop” or “latency lasso” i.e. the region of the LAP that is in closest association with the mature TGF-β1 within the latent complex (see Shi et al. supra), is located at amino acid positions 59-73 of the full-length TGF-β1 protein (see SEQ ID NOs: 1 and 5) or amino acid positions 30-44 of the LAP region (see SEQ ID NOs: 3 and 7). It is noteworthy therefore, that in certain embodiments, the TGF-β1 activating antibodies or antigen binding fragments described herein bind to an epitope comprising amino acids from a region of the LAP distal to the latency loop or latency lasso. These antibodies or antigen binding fragments are, nevertheless, able to activate TGF-β1 by relieving the inhibition mediated by the LAP.

The region of TGF-β1 consisting of amino acids 201-222 differs between the human and mouse TGF-β1 proteins as shown below:

Human latent TGF-β1: (aa 201-222)
(SEQ ID NO: 80)
TGVVRQWLSRGGEIEGFRLSAH
Murine latent TGF-β1: (aa 201-222)
(SEQ ID NO: 81)
TGVVRQWLNQGDGIQGFRFSAH.

In certain embodiments, the activating TGF-β1 antibodies or antigen binding fragments that bind to human latent TGF-β1 bind to an epitope comprising amino acid residues or to a conformational epitope requiring amino acid residues E119, K123, S138, R141, S209, R210, G212, E213, 1214, and/or E215 of the full length TGF-β1 protein as represented by SEQ ID NO: 1.

In certain embodiments, the activating TGF-β1 antibodies or antigen binding fragments that bind to human latent TGF-31 interfaces to a region comprising amino acid residues or to a conformational epitope requiring amino acid residues D122, F124, F134, E142, P145, E146, P147, K173, Y174, S175, N176, and/or N177 of the full length TGF-1 protein as represented by SEQ ID NO: 1.

In certain embodiments, the activating TGF-β1 antibodies or antigen binding fragments that bind to human latent TGF-β1 bind to an epitope comprising amino acid E119 and/or K123 of the full length TGF-β1 protein as represented by SEQ ID NO: 1. For antibodies or antigen binding fragments that bind to an epitope comprising E119 and/or K123 of human latent TGF-β1, in certain embodiments, the epitope does not comprise another amino acid residue from within the region of human latent TGF-β1 consisting of amino acids 87-135.

In certain embodiments, the activating TGF-β1 antibodies or antigen binding fragments that bind to human latent TGF-β1 bind to a conformational epitope requiring amino acid residues E119 and/or K123 of the full length TGF-β1 protein as represented by SEQ ID NO: 1. For antibodies or antigen binding fragments that bind to a conformational epitope requiring E119 and/or K123 of human latent TGF-β1, in certain embodiments, the epitope does not require another amino acid residue from within the region of human latent TGF-β1 consisting of amino acids 87-135.

In certain embodiments, the activating TGF-β1 antibodies or antigen binding fragments that bind to human latent TGF-β1 interfaces to a region comprising amino acid residues or to a conformational epitope requiring amino acid residues D122, F124 and/or F134 of the full length TGF-β1 protein as represented by SEQ ID NO: 1, within the region of human latent TGF-β1 consisting of amino acids 87-135.

In certain embodiments, the activating TGF-β1 antibodies or antigen binding fragments that bind to human latent TGF-β1 bind to an epitope comprising amino acid S138 and/or R141 of the full length TGF-β1 protein as represented by SEQ ID NO: 1. For antibodies or antigen binding fragments that bind to an epitope comprising S138 and/or R141 of human latent TGF-β1, in certain embodiments, the epitope does not comprise another amino acid residue from within the region of human latent TGF-β1 consisting of amino acids 136-200.

In certain embodiments, the activating TGF-β1 antibodies or antigen binding fragments that bind to human latent TGF-β1 bind to a conformational epitope requiring amino acid residues S138 and/or R141 of the full length TGF-β1 protein as represented by SEQ ID NO: 1. For antibodies or antigen binding fragments that bind to a conformational epitope requiring S138 and/or R141 of human latent TGF-β1, in certain embodiments, the epitope does not require another amino acid residue from within the region of human latent TGF-β1 consisting of amino acids 136-200.

In certain embodiments, the activating TGF-β1 antibodies or antigen binding fragments that bind to human latent TGF-β1 interfaces to a region comprising amino acid residues or to a conformational epitope requiring amino acid residues E142, P145, E146, P147, K173, Y174, S175, N176, and/or N177 of the full length TGF-β1 protein as represented by SEQ ID NO: 1, within the region of human latent TGF-β1 consisting of amino acids 136-200.

In certain embodiments, the activating TGF-β1 antibodies or antigen binding fragments that bind to human latent TGF-β1 bind to an epitope comprising amino acid residues S209, R210, G212, E213, 1214 and/or E215 of the full length TGF-β1 protein as represented by SEQ ID NO: 1. For antibodies or antigen binding fragments that bind to an epitope comprising S209, R210, G212, E213, 1214 and/or E215 of human latent TGF-β1, in certain embodiments, the epitope does not comprise another amino acid residue from within the region of human latent TGF-β1 consisting of amino acids 201-222. In certain embodiments, the activating TGF-β1 antibodies or antigen binding fragments do not bind to R205 and/or R210 and/or E213.

In certain embodiments, the activating TGF-β1 antibodies or antigen binding fragments that bind to human latent TGF-31 bind to a conformational epitope requiring amino acid residues S209, R210, G212, E213, 1214 and/or E215 of the full length TGF-β1 protein as represented by SEQ ID NO: 1. For antibodies or antigen binding fragments that bind to a conformational epitope requiring S209, R210, G212, E213, 1214 and/or E215 of human latent TGF-β1, in certain embodiments, the epitope does not require another amino acid residue from within the region of human latent TGF-31 consisting of amino acids 201-222. In certain embodiments, the activating TGF-β1 antibodies or antigen binding fragments bind to a conformational epitope that does not require R205 and/or R210 and/or E213.

In certain embodiments, the activating TGF-β1 antibodies or antigen binding fragments that bind to human latent TGF-β1 interfaces to a region comprising amino acid residues or to a conformational epitope requiring amino acid residues L208 and/or G211 of the full length TGF-β1 protein as represented by SEQ ID NO: 1, within the region of human latent TGF-β1 consisting of amino acids 201-222. In one embodiment, the region comprising amino acid residues or to a conformational epitope requiring amino acid residues L208 and/or G211 of the full length TGF-β1 protein as represented by SEQ ID NO: 1 are required for the activity of the activating TGF-β1 antibodies or antigen binding fragments thereof.

In certain embodiments, the amino acid residues of human latent TGF-1 protein as represented by SEQ ID NO: 1 that are required for the activity of the activating TGF-β1 antibodies or antigen binding fragments comprise G212 and E215. In certain embodiments, the amino acid residues of human latent TGF-β1 protein as represented by SEQ ID NO: 1 that are required for the activity of the activating TGF-β1 antibodies or antigen binding fragments comprise W207, L208, G211, G212, 1214, E215 and F217.

In certain embodiments, the activating TGF-β1 antibodies or antigen binding fragments that bind to murine latent TGF-β1 bind to an epitope comprising amino acid residues or to a conformational epitope requiring amino acid residues S138, R141, P145, E146, S175, L208, N209 and/or D212 of the full length TGF-β1 protein as represented by SEQ ID NO: 5.

In certain embodiments, the activating TGF-β1 antibodies or antigen binding fragments that bind to murine latent TGF-β1 bind to an epitope comprising amino acid residues S138, R141, P145, E146 and/or S175 of the full length TGF-1 protein as represented by SEQ ID NO: 5. For antibodies or antigen binding fragments that bind to an epitope comprising S138, R141, P145, E146 and/or S175 of murine latent TGF-β1, in certain embodiments, the epitope does not comprise another amino acid residue from within the region of murine latent TGF-β1 consisting of amino acids 136-200. In certain embodiments, the activating TGF-β1 antibodies or antigen binding fragments do not bind to G211 and/or F217.

In certain embodiments, the activating TGF-β1 antibodies or antigen binding fragments that bind to murine latent TGF-β1 bind to a conformational epitope requiring amino acid S138, R141, P145, E146 and/or S175 of the full length TGF-β1 protein as represented by SEQ ID NO: 5. For antibodies or antigen binding fragments that bind to a conformational epitope S138, R141, P145, E146 and/or S175 of murine latent TGF-β1, in certain embodiments, the epitope does not require another amino acid residue from within the region of murine latent TGF-β1 consisting of amino acids 136-200. In certain embodiments, the activating TGF-β1 antibodies or antigen binding fragments bind to a conformational epitope that does not require G211 and/or F217.

In certain embodiments, the activating TGF-1 antibodies or antigen binding fragments that bind to murine latent TGF-β1 interfaces to a region comprising amino acid residues or to a conformational epitope requiring amino acid residues E142, P147, P148, L149, Y174 and/or N176 of the full length TGF-1 protein as represented by SEQ ID NO: 5, within the region of murine latent TGF-β1 consisting of amino acids 136-200.

In certain embodiments, the activating TGF-β1 antibodies or antigen binding fragments that bind to murine latent TGF-31 bind to an epitope comprising amino acid residues L208, N209 and D212 of the full length TGF-β1 protein as represented by SEQ ID NO: 5. For antibodies or antigen binding fragments that bind to an epitope comprising L208, N209 and D212 of murine latent TGF-β1, in certain embodiments, the epitope does not comprise another amino acid residue from within the region of murine latent TGF-1 consisting of amino acids 201-222. In certain embodiments, the activating TGF-β1 antibodies or antigen binding fragments do not bind to G211 and/or F217.

In certain embodiments, the activating TGF-β1 antibodies or antigen binding fragments that bind to murine latent TGF-β1 bind to a conformational epitope requiring amino acid residues L208, N209 and D212 of the full length TGF-β1 protein as represented by SEQ ID NO: 5. For antibodies or antigen binding fragments that bind to a conformational epitope requiring L208, N209 and D212 of murine latent TGF-β1, in certain embodiments, the epitope does not require another amino acid residue from within the region of murine latent TGF-β1 consisting of amino acids 201-222. In certain embodiments, the activating TGF-β1 antibodies or antigen binding fragments bind to a conformational epitope that does not require G211 and/or F217.

In certain embodiments, the activating TGF-β1 antibodies or antigen binding fragments that bind to murine latent TGF-β1 interfaces to a region comprising amino acid residues or to a conformational epitope requiring amino acid residues L208, G211, of the full length TGF-β1 protein as represented by SEQ ID NO: 5, within the region of murine latent TGF-β1 consisting of amino acids 201-222.

In certain embodiments, the amino acid residues of murine latent TGF-β1 protein as represented by SEQ ID NO: 5 that are required for the activity of the activating TGF-β1 antibodies or antigen binding fragments comprise L208 and/or D212. In certain embodiments, the amino acid residues of murine latent TGF-1 protein as represented by SEQ ID NO: 5 that are required for the activity of the activating TGF-β1 antibodies or antigen binding fragments comprise L208 and/or D212.

### Antibody and Antigen Binding Fragment Format

The TGF-β1 antibodies and antigen binding fragments of the present invention may adopt any suitable form. The term “antibody” herein is used in the broadest sense and encompasses, but is not limited to, monoclonal antibodies (including full length monoclonal antibodies), polyclonal antibodies, multispecific antibodies (e.g., bispecific antibodies), VHH antibodies, so long as they exhibit the appropriate immunological specificity for latent TGF-β1.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes) on the antigen, each monoclonal antibody is directed against a single determinant or epitope on the antigen.

“Antibody fragments” or “antigen binding fragments” comprise a portion of a full-length antibody, generally the antigen binding or variable domain thereof. Antibody fragments are described elsewhere herein and examples of antibody fragments include Fab, Fab′, F(ab′)2, bi-specific Fabs, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

In certain embodiments, the TGF-β1 antibodies or antigen binding fragments of the invention are multivalent, preferably bivalent. In certain embodiments, the TGF-β1 antibodies or antigen binding fragments are bivalent having two antigen binding sites that are identical and that immuno-react with the same epitope on latent TGF-β1. Such bivalent antibodies and antigen binding fragments include but are not limited to conventional heterotetrameric Y-shaped antibodies, F(ab′)2 fragments and (scFv)2 fragments.

The TGF-β1 antibodies and antigen binding fragments described herein are intended for human therapeutic use and therefore, will typically be immunoglobulins of the IgA, IgD, IgE, IgG, IgM type, often of the IgG type, in which case they can belong to any of the four sub-classes IgG1, IgG2a and b, IgG3 or IgG4. In preferred embodiments, the TGF-β1 antibodies are IgG antibodies. Particularly preferred are IgG1 antibodies. Monoclonal antibodies are preferred since they are highly specific, being directed against a single antigenic site.

The TGF-β1 antibodies and antigen binding fragments may exhibit high human homology as defined elsewhere herein. Such antibody molecules having high human homology may include antibodies comprising VH and VL domains of native non-human antibodies which exhibit sufficiently high % sequence identity to human germline sequences. In certain embodiments, the antibodies or antigen binding fragments thereof are humanised or germlined variants of non-human antibodies.

In certain embodiments, the TGF-β1 antibodies and antigen binding fragments described herein are camelid-derived. Camelid-derived antibodies may be heavy-chain only antibodies i.e. VHH antibodies or may be conventional heterotetrameric antibodies. In preferred embodiments, the TGF-β1 and antigen binding fragments are derived from camelid heterotetrameric antibodies. In further preferred embodiments, the TGF-β1 antibodies are VHH antibodies or are derived from VHH antibodies.

For example, the TGF-β1 antibodies and antigen binding fragments may be selected from immune libraries obtained by a method comprising the step of immunizing a camelid with the target of interest. The camelid may be immunized with the target protein or polypeptide fragment thereof, or with an mRNA molecule or cDNA molecule expressing the protein or a polypeptide fragment thereof. Methods for producing antibodies in camelid species and selecting antibodies against preferred targets from camelid immune libraries are described in, for example, International patent application no. WO2010/001251, incorporated herein by reference.

In certain embodiments, the TGF-β1 antibodies and antigen binding fragments may be camelid-derived in that they comprise at least one hypervariable (HV) loop or complementarity determining region obtained from a VH domain or a VL domain of a species in the family Camelidae. In particular, the TGF-β1 antibodies and antigen binding fragments may comprise VH and/or VL domains, or CDRs thereof, obtained by active immunisation of outbred camelids, e.g. llamas, with latent TGF-β1.

The term “obtained from” in this context implies a structural relationship, in the sense that the HVs or CDRs of the antibodies embody an amino acid sequence (or minor variants thereof) which was originally encoded by a Camelidae immunoglobulin gene. However, this does not necessarily imply a particular relationship in terms of the production process used to prepare the antibodies or antigen binding fragments thereof.

Camelid-derived antibodies or antigen binding fragments thereof may be derived from any camelid species, including inter alia, llama, dromedary, alpaca, vicuna, guanaco or camel.

Antibody molecules comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. camelid and human.

In certain embodiments, the entire VH domain and/or the entire VL domain may be obtained from a species in the family Camelidae. The camelid-derived VH domain and/or the camelid-derived VL domain may then be subject to protein engineering, in which one or more amino acid substitutions, insertions or deletions are introduced into the camelid amino acid sequence. These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain.

Isolated camelid VH and VL domains obtained by active immunisation of a camelid (e.g. llama) with latent TGF-β1 can be used as a basis for engineering TGF-β1 antibodies and antigen binding fragments in accordance with the present invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain.

In other embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments, both the VH and the VL domain may be derived from a single animal, particularly a single animal which has been actively immunised with the antigen of interest.

As an alternative to engineering changes in the primary amino acid sequence of Camelidae VH and/or VL domains, individual camelid-derived hypervariable loops or CDRs, or combinations thereof, can be isolated from camelid VH/VL domains and transferred to an alternative (i.e. non-Camelidae) framework, e.g. a human VH/VL framework, by CDR grafting.

In non-limiting embodiments, the TGF-β1 antibodies may comprise CH1 domains and/or CL domains (from the heavy chain and light chain, respectively), the amino acid sequence of which is fully or substantially human. For antibody molecules intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence. The CH1 domain, hinge region, CH2 domain, CH3 domain and/or CL domain (and/or CH4 domain if present) may be derived from a human antibody, preferably a human IgG antibody, more preferably a human IgG1 antibody of subtype IgG1, IgG2, IgG3 or IgG4.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required.

The TGF-β1 antibodies may have one or more amino acid substitutions, insertions or deletions within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites).

The TGF-β1 antibodies may be modified within the Fc region to increase binding affinity for the neonatal receptor FcRn. The increased binding affinity may be measurable at acidic pH (for example from about approximately pH 5.5 to approximately pH 6.0). The increased binding affinity may also be measurable at neutral pH (for example from approximately pH 6.9 to approximately pH 7.4). By “increased binding affinity” is meant increased binding affinity to FcRn relative to the unmodified Fc region. Typically the unmodified Fc region will possess the wild-type amino acid sequence of human IgG1, IgG2, IgG3 or IgG4. In such embodiments, the increased FcRn binding affinity of the antibody molecule having the modified Fc region will be measured relative to the binding affinity of wild-type IgG1, IgG2, IgG3 or IgG4 for FcRn.

In certain embodiments, one or more amino acid residues within the Fc region may be substituted with a different amino acid so as to increase binding to FcRn. Several Fc substitutions have been reported that increase FcRn binding and thereby improve antibody pharmacokinetics. Such substitutions are reported in, for example, Zalevsky et al. (2010) Nat. Biotechnol. 28 (2): 157-9; Hinton et al. (2006) J Immunol. 176:346-356; Yeung et al. (2009) J Immunol. 182:7663-7671; Presta L G. (2008) Curr. Op. Immunol. 20:460-470; and Vaccaro et al. (2005) Nat. Biotechnol. 23 (10): 1283-88, the contents of which are incorporated herein in their entirety.

In certain embodiments, the TGF-β1 antibodies comprise a modified human IgG Fc domain comprising or consisting of the amino acid substitutions H433K and N434F, wherein the Fc domain numbering is in accordance with EU numbering. In a further embodiment, the TGF-β1 antibodies described herein comprise a modified human IgG Fc domain comprising or consisting of the amino acid substitutions M252Y, S254T, T256E, H433K and N434F, wherein the Fc domain numbering is in accordance with EU numbering.

In certain embodiments, the TGF-β1 antibodies comprise a modified human IgG Fc domain consisting of up to 2, up to 3, up to 4, up to 5, up to 6, up to 7, up to 8, up to 9, up to 10, up to 12, up to 15, or up to 20 substitutions relative to the corresponding wild-type IgG sequence.

In certain embodiments, the Fc region may be engineered such that there is no effector function. In certain embodiments, the antibodies of the invention may have an Fc region derived from naturally-occurring IgG isotypes having reduced effector function, for example IgG4. Fc regions derived from IgG4 may be further modified to increase therapeutic utility, for example by the introduction of modifications that minimise the exchange of arms between IgG4 molecules in vivo. Fc regions derived from IgG4 may be modified to include the S228P substitution.

In certain embodiments, one, two, or more amino acid substitutions are introduced into an IgG constant region Fc region to alter the effector function(s) of the polypeptide. For example, one or more of the following mutations in the constant region of the antibodies described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; an A330L substitution; an 1332E substitution; or a P396L substitution, numbered according to the EU numbering system.

In certain embodiments, one or more of the following mutations are introduced into the constant region of the antibodies described herein: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; an A330L substitution; an 1332E substitution; or a P396L substitution,

Such an antibody with altered, diminished or even abolished effector function is attractive in the context of the invention.

In certain embodiments, the antibodies and antigen binding fragments are modified with respect to glycosylation. For example, an aglycoslated antibody can be made (i.e., the antibody lacks glycosylation). Glycosylation can be altered to, for example, increase the affinity of the antibody for the target antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

### C. Exemplary Activating TGF-31 Antibodies that Bind Latent TGF-31

The present invention provides exemplary TGF-β1 antibodies and antigen binding fragments thereof. The exemplary TGF-β1 antibodies and antigen binding fragments of the invention may be defined exclusively with respect to their structural characteristics, as described below.

In some embodiments, the invention includes antibody or antigen binding fragment thereof wherein the CDR have an amino acid sequence that shares at least about 90% of identity with SEQ ID NOs described herein, or at least 91%, at least 92%, at least 93%, at least 94%, at least 95%, at least 96%, at least 97%, at least 97%, at least 99%, at least 99.5%, or at least 99.9%.

In certain embodiments, there is provided an antibody or antigen binding fragment thereof, which binds to human latent TGF-β1, said antibody or antigen binding fragment comprising a heavy chain variable domain and a light chain variable domain, wherein
the variable heavy chain CDR3 sequence comprises or consists of SEQ ID NO:11 [GGSIDLTYGMDY] or sequence variant thereof;
the variable heavy chain CDR2 sequence comprises or consists SEQ ID NO:10 [AIRWNGVTTYYAESMKG] or sequence variant thereof;
the variable heavy chain CDR1 sequence comprises or consists of SEQ ID NO:9 [DYTMN] or sequence variant thereof;
the variable light chain CDR3 sequence comprises or consists of SEQ ID NO:26 [ALYMYTGSNNGRV] or sequence variant thereof;
the variable light chain CDR2 sequence comprises or consists SEQ ID NO:25 [NTVARHS] or sequence variant thereof;
the variable light chain CDR1 sequence comprises or consists of SEQ ID NO:24 [GLSSGSVTRNNYPD] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to human latent TGF-β1, said antibody or antigen binding fragment comprising a heavy chain variable domain and a light chain variable domain, wherein
the variable heavy chain CDR3 sequence comprises or consists of SEQ ID NO:14 [SRYGVVITTTYFGGVDY] or sequence variant thereof;
the variable heavy chain CDR2 sequence comprises or consists SEQ ID NO:13 [AINTGGGITYYTDSVKG] or sequence variant thereof;
the variable heavy chain CDR1 sequence comprises or consists of SEQ ID NO:12 [HYWMY] or sequence variant thereof;
the variable light chain CDR3 sequence comprises or consists of SEQ ID NO:29 [LSYESSGYAV] or sequence variant thereof;
the variable light chain CDR2 sequence comprises or consists SEQ ID NO:28 [GNSNRPS] or sequence variant thereof;
the variable light chain CDR1 sequence comprises or consists of SEQ ID NO:27 [QGGNFGSYYAS] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to human latent TGF-β1, said antibody or antigen binding fragment comprising a heavy chain variable domain and a light chain variable domain, wherein
the variable heavy chain CDR3 sequence comprises or consists of SEQ ID NO:57 [YITRAQLMGAWDY] or sequence variant thereof;
the variable heavy chain CDR2 sequence comprises or consists SEQ ID NO:56 [RITNSGGTAFYTDSVEG] or sequence variant thereof;
the variable heavy chain CDR1 sequence comprises or consists of SEQ ID NO:55 [SYWMD] or sequence variant thereof;
the variable light chain CDR3 sequence comprises or consists of SEQ ID NO:60 [AQSTYYPYD] or sequence variant thereof;
the variable light chain CDR2 sequence comprises or consists SEQ ID NO:59 [QVSNRAS] or sequence variant thereof;
the variable light chain CDR1 sequence comprises or consists of SEQ ID NO:58 [KASQSLVHTDGKTYLS] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to human latent TGF-β1, said antibody or antigen binding fragment comprising a heavy chain variable domain and a light chain variable domain, wherein
the variable heavy chain CDR3 sequence comprises or consists of SEQ ID NO:11 [GGSIDLTYGMDY] or sequence variant thereof, or SEQ ID NO:14 [SRYGVVITTTYFGGVDY] or sequence variant thereof, or SEQ ID NO:57 [YITRAQLMGAWDY] or sequence variant thereof;
the variable heavy chain CDR2 sequence comprises or consists SEQ ID NO:10 [AIRWNGVTTYYAESMKG] or sequence variant thereof, or SEQ ID NO:13 [AlNTGGGITYYTDSVKG] or sequence variant thereof, or SEQ ID NO:56 [RITNSGGTAFYTDSVEG] or sequence variant thereof;
the variable heavy chain CDR1 sequence comprises or consists of SEQ ID NO:9 [DYTMN] or sequence variant thereof, or SEQ ID NO:12 [HYWMY] or sequence variant thereof, or SEQ ID NO: 55 [SYWMD] or sequence variant thereof;
the variable light chain CDR3 sequence comprises or consists of SEQ ID NO:26 [ALYMYTGSNNGRV] or sequence variant thereof, or SEQ ID NO:29 [LSYESSGYAV] or sequence variant thereof, or SEQ ID NO:60 [AQSTYYPYD] or sequence variant thereof;
the variable light chain CDR2 sequence comprises or consists SEQ ID NO:25 [NTVARHS] or sequence variant thereof, or SEQ ID NO:28 [GNSNRPS] or sequence variant thereof, or SEQ ID NO: 59 [QVSNRAS] or sequence variant thereof;
the variable light chain CDR1 sequence comprises or consists of SEQ ID NO:24 [GLSSGSVTRNNYPD] or sequence variant thereof, or SEQ ID NO:27 [QGGNFGSYYAS] or sequence variant thereof, or SEQ ID NO:58 [KASQSLVHTDGKTYLS] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to human latent TGF-β1, said antibody or antigen binding fragment comprising a heavy chain variable domain and a light chain variable domain, wherein
the variable heavy chain CDR3 sequence comprises or consists of SEQ ID NO:11 [GGSIDLTYGMDY] or sequence variant thereof, or SEQ ID NO:14 [SRYGVVITTTYFGGVDY] or sequence variant thereof;
the variable heavy chain CDR2 sequence comprises or consists SEQ ID NO:10 [AIRWNGVTTYYAESMKG] or sequence variant thereof, or SEQ ID NO:13 [AlNTGGGITYYTDSVKG] or sequence variant thereof;
the variable heavy chain CDR1 sequence comprises or consists of SEQ ID NO:9 [DYTMN] or sequence variant thereof, or SEQ ID NO:12 [HYWMY] or sequence variant thereof;
the variable light chain CDR3 sequence comprises or consists of SEQ ID NO:26 [ALYMYTGSNNGRV] or sequence variant thereof, or SEQ ID NO:29 [LSYESSGYAV] or sequence variant thereof;
the variable light chain CDR2 sequence comprises or consists SEQ ID NO:25 [NTVARHS] or sequence variant thereof, or SEQ ID NO:28 [GNSNRPS] or sequence variant thereof;
the variable light chain CDR1 sequence comprises or consists of SEQ ID NO:24 [GLSSGSVTRNNYPD] or sequence variant thereof, or SEQ ID NO:27 [QGGNFGSYYAS] or sequence variant thereof; and wherein the sequence variant comprises one, two or three amino acid alterations (e.g., substitutions (e.g., conservative substitutions, humanising substitutions, affinity variant substitutions), additions, or deletions) in the recited sequence.

In certain embodiments, the antibodies and antigen binding fragments that bind to human latent TGF-β1 comprise or consist of a variable heavy chain domain (VH) and a variable light chain domain (VL), wherein a VH comprises or consists of the amino acid sequence of SEQ ID NO: 37 or an amino acid sequence at least 90%, 95%, 97%, 98% or 99% identical thereto and a VL comprises or consists of the amino acid sequence of SEQ ID NO: 38 or an amino acid sequence at least 90%, 95%, 97%, 98% or 99% identical thereto.

In certain embodiments, the antibodies and antigen binding fragments that bind to human latent TGF-β1 comprise or consist of a variable heavy chain domain (VH) and a variable light chain domain (VL), wherein a VH comprises or consists of the amino acid sequence of SEQ ID NO: 39 or an amino acid sequence at least 90%, 95%, 97%, 98% or 99% identical thereto and a VL comprises or consists of the amino acid sequence of SEQ ID NO: 40 or an amino acid sequence at least 90%, 95%, 97%, 98% or 99% identical thereto.

In certain embodiments, the antibodies and antigen binding fragments that bind to human latent TGF-β1 comprise or consist of a variable heavy chain domain (VH) and a variable light chain domain (VL), wherein a VH comprises or consists of the amino acid sequence of SEQ ID NO: 61 or an amino acid sequence at least 90%, 95%, 97%, 98% or 99% identical thereto and a VL comprises or consists of the amino acid sequence of SEQ ID NO: 62 or an amino acid sequence at least 90%, 95%, 97%, 98% or 99% identical thereto.

In certain embodiments, the antibodies and antigen binding fragments that bind to human latent TGF-β1 are selected from antibodies and antigen binding fragments comprising or consisting of a variable heavy chain domain (VH) and a variable light chain domain (VL) selected from the group consisting of:
(i) a VH comprising or consisting of the amino acid sequence of SEQ ID NO: 37 or an amino acid sequence at least 90%, 95%, 97%, 98% or 99% identical thereto and a VL comprising or consisting of the amino acid sequence of SEQ ID NO: 38 or an amino acid sequence at least 90%, 95%, 97%, 98% or 99% identical thereto;
(ii) a VH comprising or consisting of the amino acid sequence of SEQ ID NO: 39 or an amino acid sequence at least 90%, 95%, 97%, 98% or 99% identical thereto and a VL comprising or consisting of the amino acid sequence of SEQ ID NO: 40 or an amino acid sequence at least 90%, 95%, 97%, 98% or 99% identical thereto; and
(iii) VH comprising or consisting of the amino acid sequence of SEQ ID NO: 61 or an amino acid sequence at least 90%, 95%, 97%, 98% or 99% identical thereto and a VL comprising or consisting of the amino acid sequence of SEQ ID NO: 62 or an amino acid sequence at least 90%, 95%, 97%, 98% or 99% identical thereto.

In certain embodiments, the antibodies and antigen binding fragments that bind to human latent TGF-β1 are selected from antibodies and antigen binding fragments comprising or consisting of a variable heavy chain domain (VH) and a variable light chain domain (VL) selected from the group consisting of:
(i) a VH comprising or consisting of the amino acid sequence of SEQ ID NO: 37 or an amino acid sequence at least 90%, 95%, 97%, 98% or 99% identical thereto and a VL comprising or consisting of the amino acid sequence of SEQ ID NO: 38 or an amino acid sequence at least 90%, 95%, 97%, 98% or 99% identical thereto; and
(ii) a VH comprising or consisting of the amino acid sequence of SEQ ID NO: 39 or an amino acid sequence at least 90%, 95%, 97%, 98% or 99% identical thereto and a VL comprising or consisting of the amino acid sequence of SEQ ID NO: 40 or an amino acid sequence at least 90%, 95%, 97%, 98% or 99% identical thereto.

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions.

In certain embodiments, there is provided an antibody or antigen binding fragment thereof, which binds to murine latent TGF-β1, said antibody or antigen binding fragment comprising a heavy chain variable domain and a light chain variable domain, wherein
the variable heavy chain CDR3 sequence comprises or consists of SEQ ID NO:17 [DRIRSSYEYDS] or sequence variant thereof;
the variable heavy chain CDR2 sequence comprises or consists SEQ ID NO:16 [HISAAGISRYYADAVKG] or sequence variant thereof;
the variable heavy chain CDR1 sequence comprises or consists of SEQ ID NO:15 [NYGVH] or sequence variant thereof;
the variable light chain CDR3 sequence comprises or consists of SEQ ID NO:32 [GCYDSSLSTGYV] or sequence variant thereof;
the variable light chain CDR2 sequence comprises or consists SEQ ID NO:31 [NANNRAS] or sequence variant thereof;
the variable light chain CDR1 sequence comprises or consists of SEQ ID NO:30 [TGSSSNIGGGYYLS] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to murine latent TGF-β1, said antibody or antigen binding fragment comprising a heavy chain variable domain and a light chain variable domain, wherein
the variable heavy chain CDR3 sequence comprises or consists of SEQ ID NO:20 [GLVSSGLNGMDY] or sequence variant thereof;
the variable heavy chain CDR2 sequence comprises or consists SEQ ID NO:19 [AITPGGTATYYANSVKG] or sequence variant thereof;
the variable heavy chain CDR1 sequence comprises or consists of SEQ ID NO:18 [RSGMA] or sequence variant thereof;
the variable light chain CDR3 sequence comprises or consists of SEQ ID NO:34 [GCYDDSLNTYV] or sequence variant thereof;
the variable light chain CDR2 sequence comprises or consists SEQ ID NO:33 [NTNNRAS] or sequence variant thereof;
the variable light chain CDR1 sequence comprises or consists of SEQ ID NO:30 [TGSSSNIGGGYYLS] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, there is provided an antibody or antigen binding fragment thereof, which binds to murine latent TGF-β1, said antibody or antigen binding fragment comprising a heavy chain variable domain and a light chain variable domain, wherein
the variable heavy chain CDR3 sequence comprises or consists of SEQ ID NO:23 [DLNSRSYKGMGD] or sequence variant thereof;
the variable heavy chain CDR2 sequence comprises or consists SEQ ID NO:22 [GTSIGGGSTWYADSVKG] or sequence variant thereof;
the variable heavy chain CDR1 sequence comprises or consists of SEQ ID NO:21 [SYYMY] or sequence variant thereof;
the variable light chain CDR3 sequence comprises or consists of SEQ ID NO:36 [GCYDSSLSSVV] or sequence variant thereof;
the variable light chain CDR2 sequence comprises or consists SEQ ID NO:31 [NANNRAS] or sequence variant thereof;
the variable light chain CDR1 sequence comprises or consists of SEQ ID NO:35 [TGSSSNIGGGYYLN] or sequence variant thereof; and
wherein the sequence variant comprises one, two or three amino acid alterations (e.g., substitutions (e.g., conservative substitutions, humanising substitutions, affinity variant substitutions), additions, or deletions) in the recited sequence.

In certain embodiments, the antibodies and antigen binding fragments that bind to human latent TGF-β1 are selected from antibodies and antigen binding fragments comprising or consisting of a variable heavy chain domain (VH) and a variable light chain domain (VL) selected from the group consisting of:
(i) a VH comprising or consisting of the amino acid sequence of SEQ ID NO: 41 or an amino acid sequence at least 90%, 95%, 97%, 98% or 99% identical thereto and a VL comprising or consisting of the amino acid sequence of SEQ ID NO: 42 or an amino acid sequence at least 90%, 95%, 97%, 98% or 99% identical thereto;
(ii) a VH comprising or consisting of the amino acid sequence of SEQ ID NO: 43 or an amino acid sequence at least 90%, 95%, 97%, 98% or 99% identical thereto and a VL comprising or consisting of the amino acid sequence of SEQ ID NO: 44 or an amino acid sequence at least 90%, 95%, 97%, 98% or 99% identical thereto; and
(iii) a VH comprising or consisting of the amino acid sequence of SEQ ID NO: 45 or an amino acid sequence at least 90%, 95%, 97%, 98% or 99% identical thereto and a VL comprising or consisting of the amino acid sequence of SEQ ID NO: 46 or an amino acid sequence at least 90%, 95%, 97%, 98% or 99% identical thereto.

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions.

The invention also provides antibodies or antigen binding fragments thereof, which bind to the same epitope as the TGF-β1 antibodies exemplified herein.

The exemplary TGF-β1 antibodies and antigen binding fragments described herein are TGF-β1 activating antibodies i.e. they bind to latent TGF-β1 and release the mature TGF-β1 cytokine from the inhibition mediated by the LAP. The activating properties of the exemplary TGF-β1 antibodies and antigen binding fragments may be measured by any of the methods described in Section B above. Moreover, the exemplary TGF-β1 antibodies and antigen binding fragments described herein may adopt any of the antibody formats according to the embodiments described in Section B above.

In preferred embodiments, the exemplary TGF-β1 antibody molecules having the CDR sequences recited above exhibit high human homology, for example are humanised or germlined variants of the antibodies or antigen binding fragments thereof from which the CDR sequences derive.

In non-limiting embodiments, the exemplary TGF-β1 antibodies and antigen binding fragments thereof having the CDR, VH and/or VL sequences described herein may comprise CH1 domains and/or CL domains (from the heavy chain and light chain, respectively), the amino acid sequence of which is fully or substantially human. For antibody molecules intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required. Any of the exemplary Fc region modifications described herein may be incorporated into the TGF-β1 antibodies having the CDR and/or VH/VL domain sequences recited above. In certain embodiments, the TGF-β1 antibodies having the CDR and/or VH/VL domain sequences recited above comprise a modified human IgG Fc domain comprising or consisting of the amino acid substitutions H433K and N434F, wherein the Fc domain numbering is in accordance with EU numbering. In certain embodiments, the TGF-β1 antibodies having the CDR and/or VH/VL domain sequences recited above comprise a modified human IgG Fc domain comprising or consisting of the amino acid substitutions M252Y, S254T, T256E, H433K and N434F.

Unless otherwise stated in the present application, % sequence identity between two amino acid sequences may be determined by comparing these two sequences aligned in an optimum manner and in which the amino acid sequence to be compared can comprise additions or deletions with respect to the reference sequence for an optimum alignment between these two sequences. The percentage of identity is calculated by determining the number of identical positions for which the amino acid residue is identical between the two sequences, dividing this number of identical positions by the total number of positions in the comparison window and multiplying the result obtained by 100 in order to obtain the percentage of identity between these two sequences. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/bl2.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

For embodiments wherein the domains of the antibodies or antigen binding fragments are defined by a particular percentage sequence identity to a reference sequence, the VH, VHH and/or VL domains may retain identical CDR sequences to those present in the reference sequence such that the variation is present only within the framework regions.

### D. Nucleic Acids Encoding TGF-β1 Activating Antibodies

The invention also provides polynucleotide molecules or nucleic acids encoding the TGF-β1 antibodies or antigen binding fragments of the invention. Polynucleotide molecules or nucleic acids encoding the full-length antibodies are provided, together with polynucleotide molecules or nucleic acids encoding fragments or individual chains of the antibodies, for example the VH, VHH and/or VL domains of the TGF-β1 antibodies described herein. Also provided are expression vectors containing said nucleotide sequences of the invention operably linked to regulatory sequences which permit expression of the antibodies or fragments thereof in a host cell or cell-free expression system, and a host cell or cell-free expression system containing this expression vector.

Polynucleotide molecules encoding antibodies or antigen binding fragments of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

For recombinant production of an antibody according to the invention, a recombinant polynucleotide encoding it or recombinant polynucleotides encoding the different chains or domains may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NS0 (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antigen binding polypeptide according to the invention has been introduced are explicitly excluded from the definition of a “host cell”.

### E. Antibody Production

In a further aspect, the invention also provides a method of producing antibodies or antigen binding fragments of the invention which comprises culturing a host cell (or cell free expression system) containing polynucleotide or nucleic acid (e.g. an expression vector) encoding the antibody or antigen binding fragment under conditions which permit expression of the antibody or fragment, and recovering the expressed antibody. This recombinant expression process can be used for large scale production of antibodies, including activating TGF-β1 antibodies according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

### F. Pharmaceutical Compositions

The scope of the invention includes pharmaceutical compositions, containing one or a combination of antibodies or antigen binding fragments as described herein, formulated with one or more pharmaceutically acceptable carriers or excipients. Such compositions may include a combination of (i.e., two or more different) activating TGF-β1 antibodies. Techniques for formulating monoclonal antibodies for human therapeutic use are well known in the art and are reviewed, for example, in Wang et al., Journal of Pharmaceutical Sciences 2007; 96:1-26, the contents of which are incorporated herein in their entirety.

Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In certain embodiments, the compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration.

### G. Methods of Treatment

As reported in the Examples herein, TGF-β1 activating antibodies in accordance with the invention were capable of improving the outcome in a mouse model of Graft-versus-Host disease (GvHD). Without wishing to be bound by theory, this effect is likely attributable to the action of TGF-β1 as an immunosuppressive cytokine in the regulation of the immune response.

It follows from the above, that the activating TGF-β1 antibodies and antigen binding fragments described herein may be used in methods of treatment. Thus, provided herein are TGF-β1 activating antibodies or antigen binding fragments according to any of the aspects or embodiments described elsewhere herein for use as medicaments. The TGF-β1 antibodies and antigen binding fragments for use as medicaments are typically formulated as pharmaceutical compositions. Importantly, all embodiments described above in relation to the activating TGF-β1 antibodies and antigen binding fragments, are equally applicable to the methods described herein.

Provided herein is a method of suppressing a pathological immune response in a subject in need thereof, wherein the method comprises administering to the subject a therapeutically effective amount of a TGF-β1 activating antibody or antigen binding fragment according to any of the aspects or embodiments described elsewhere herein. Further provided is a method of preventing or treating an autoimmune disease in a subject in need thereof, wherein the method comprises administering to the subject a therapeutically effective amount of a TGF-β1 activating antibody or antigen binding fragment according to any of the aspects or embodiments described elsewhere herein. Further provided is a method of preventing or treating an allo-immune disease in a subject in need thereof, wherein the method comprises administering to the subject a therapeutically effective amount of a TGF-β1 activating antibody or antigen binding fragment according to any of the aspects or embodiments described elsewhere herein. The subject is typically a human subject. As used herein, the term “therapeutically effective amount” is intended to mean the quantity or dose of TGF-β1 antibody or antigen binding fragment that is sufficient to produce a therapeutic effect, for example, the quantity or dose required to eradicate or at least alleviate the symptoms associated with a disease or condition. An appropriate amount or dose can be determined by a physician, as required. For example, the dose can be adjusted based on factors such as the size or weight of a subject to be treated, the age of the subject to be treated, the general physical condition of the subject to be treated, the condition to be treated, and the route of administration.

The present invention also provides TGF-β1 activating antibodies or antigen binding fragments thereof in accordance with the invention for use in suppressing a pathological immune response in a subject in need thereof. Further provided are TGF-β1 activating antibodies or antigen binding fragments thereof in accordance with the invention for use in preventing or treating an autoimmune disease in a subject in need thereof. Further provided are TGF-β1 activating antibodies or antigen binding fragments in accordance with the invention for use in preventing or treating an allo-immune disease in a subject in need thereof. The subject is typically a human subject.

Autoimmune and allo-immune diseases or conditions to be prevented or treated in accordance with the invention include but are not limited to the group consisting of: Inflammatory Bowel Disease (IBD), multiple sclerosis (MS), graft-versus-host disease (GVHD), allograft rejection, antibody-mediated allograft rejection (AMR), allogenic islet graft rejection, alopecia areata, ankylosing spondylitis, antiphospholipid syndrome (APS), autoimmune Addison's disease, Alzheimer's disease, antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura or idiopathic thrombocytopenia purpura or immune mediated thrombocytopenia), autoimmune urticaria, Behcet's disease, bullous pemphigoid (BP), cardiomyopathy, Castleman's syndrome, celiac spruce-dermatitis, chronic fatigue immune dysfunction syndrome, chronic inflammatory demyelinating polyneuropathy (CIDP), Churg-Strauss syndrome, cicatrical pemphigoid, CREST syndrome, cold agglutinin disease, COVID-19 mediated-postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barré syndrome (GBS), Goodpasture's syndrome, Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen plantus, lichen sclerosus, lupus erthematosis, systemic lupus erythematosus (SLE), lupus nephritis, membranous neuropathy, membranous nephropathy, Meniere's disease, mixed connective tissue disease, mucous membrane pemphigoid, graft type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Reynauld's phenomenon, Reiter's syndrome, rheumatoid arthritis (RA), sarcoidosis, scleroderma, Sjögren's syndrome, solid organ transplant rejection, stiff-man syndrome, takayasu arteritis, toxic epidermal necrolysis (TEN), Stevens Johnson syndrome (SJS), temporal arteristis/giant cell arteritis, thrombotic thrombocytopenia purpura, thyroid eye disease, ulcerative colitis, uveitis, warm autoimmune hemolytic anemia (WAIHA), dermatitis herpetiformis vasculitis, anti-neutrophil cytoplasmic antibody-associated vasculitides, vitiligo, and Wegner's granulomatosis.

In addition to its important role as an immunosuppressive cytokine in the regulation of the immune response, TGF-β1 plays an important role in cancer development and strategies targeting TGF-β1 are being employed in cancer therapy (Fonseca Teixeira et al., Front. Cell. Dev. Biol. 2020; 8: 605). In the context of cancer, TGF-β1 plays a complex dual role having both pro-tumoral and tumour suppressive activities. Particularly in the early stages of cancer development, TGF-β1 is thought to play an important immunosuppressive role by suppressing the proliferation of epithelial cells and pre-malignant cells. At later stages of cancer progression, tumours can acquire resistance to the cytostatic effects mediated by TGF-β1.

Given the key tumour suppressive function of TGF-β1, particularly at the early stages of cancer development, provided herein are methods of preventing or treating a neoplastic disease in a subject in need thereof, wherein the method comprises administering to the subject a therapeutically effective amount of a TGF-β1 activating antibody or antigen binding fragment according to any of the aspects or embodiments described elsewhere herein. Further provided are TGF-β1 activating antibodies or antigen binding fragments thereof in accordance with the invention for use in preventing or treating a neoplastic disease in a subject in need thereof. In the context of preventing or treating neoplastic diseases, the TGF-β1 activating antibodies or antigen binding fragments described herein may activate latent TGF-β1 associated with the surface of tumour cells at a pre-malignant stage, for example the cells of a benign neoplasm.

As used herein, a method of “preventing” a disease or condition means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced.

For clinical use, in certain embodiments, the TGF-β1 antibody as described elsewhere herein is administered to a subject as one or more doses of about 0.1 mg/kg body weight to about 20 mg/kg body weight. In certain embodiments, TGF-β1 antibody is administered to a subject in a dose of about 0.1 mg/kg body weight to about 10 mg/kg body weight. In certain embodiments, the TGF-β1 antibody is administered to a subject in a dose of about 0.5 mg/kg body weight to about 10 mg/kg body weight. In certain embodiments, the TGF-β1 antibody is administered to a subject in a dose of about 1 mg/kg body weight to about 10 mg/kg body weight.

### H. Kits

Any of the TGF-β1 activating antibodies or antigen binding fragments or pharmaceutical compositions comprising the same described herein can be packaged as a kit and optionally include instructions for use.

In some embodiments, the kits are for the implementation of the treatment methods described herein. In some embodiments, the kits are for preventing or treating allo-immune and/or autoimmune diseases or conditions.

### I. Miscellaneous

Further provided is an antibody or antigen binding fragment in accordance with the first or second aspect of the invention, or a pharmaceutical composition in accordance with the invention for the manufacture of a medicament.

Provided herein is a method of activating TGF-β1 in a subject in need thereof, wherein the method comprises administering to the subject a therapeutically effective amount of a TGF-β1 activating antibody or antigen binding fragment according to any of the aspects or embodiments described elsewhere herein.

Further provided is a method of increasing the activity TGF-β1 in a subject in need thereof, wherein the method comprises administering to the subject a therapeutically effective amount of a TGF-β1 activating antibody or antigen binding fragment according to any of the aspects or embodiments described elsewhere herein. As used herein, the term “increasing the activity TGF-β1” means increasing the activity TGF-β1 by at least 1.2-fold, at least 1.5-fold, at least 2-fold, at least 2.5-fold, at least 3-fold compared to the activity of TGF-β1 in absence of a therapeutically effective amount of a TGF-β1 activating antibody or antigen binding fragment according to the invention. The activity of TGF-β1 may be assessed by any method known in the art.

Another object is a method of downregulating the immunity in a subject in need thereof, wherein the method comprises administering to the subject a therapeutically effective amount of a TGF-β1 activating antibody or antigen binding fragment according to any of the aspects or embodiments described elsewhere herein. As used herein, the term “downregulating the immunity” means reducing the immunity of the subject by at least 1%, at least 1.5%, at least 2%, at least 2.5% compared to the immunity of the subject in absence of a therapeutically effective amount of a TGF-β1 activating antibody or antigen binding fragment according to the invention. The immunity of a subject may be assessed by any method known in the art.

As compared to the TGF-β1 antibodies of the prior art, the antibodies of the present invention may present the following advantages:
in some embodiments, the antibodies of the present invention present the capacity or ability to activate TGF-β1, or to increase the activity of TGF-β1;
in some embodiments, the antibodies of the present invention present an increased affinity for TGF-β1, as compared to the TGF-β1 antibodies of the prior art;
in some embodiments, the antibodies of the present invention present an increased avidity for TGF-β1, as compared to the TGF-β1 antibodies of the prior art.

### EXAMPLES

The invention will be further understood with reference to the following non-limiting examples.

### Example 1: Fourteen Monoclonal Antibodies that Activate Latent TGF-β1

We screened a total of 134 monoclonal antibodies (mAbs) directed against human or murine GARP, GARP: (latent) TGF-β1 complexes or latent TGF-β1 for their ability to activate TGF-β1 in a TGF-β reporter assay. HEK-293T cells, which do not express GARP and secrete very low levels of soluble latent TGF-β1, were co-transfected with i) plasmids encoding GARP and latent TGF-β1 to express high levels of surface GARP: TGF-β1, and ii) a reporter plasmid encoding Firefly luciferase under the control of a TGF-β1 responsive promoter (pCAGA-12) and a control plasmid encoding Renilla luciferase under a constitutive promoter (pRL-TK) as a control for transfection efficacy. Transfected cells were incubated in the presence of the mAbs (20 μg/ml) or with serial dilutions of recombinant human active-TGF-β1 (rhTGF-β1), as a positive control. Firefly and Renilla luciferase activities were measured after 24 hours. Ratios of Firefly to Renilla luminescence measured in the absence of rhTGF-β1 or mAb were taken as a reference negative signal. Firefly/Renilla ratios superior to mean+2 SD of the negative signal were considered as positive signals for TGF-β1 activity. Fourteen of the 134 mAbs tested activated latent TGF-β1 in these assays (see FIGS. 1A and 1B). The 14 TGF-β1-activating mAbs were directed against either human latent TGF-β1 (6 of 24 mAbs called LHT-1 to -24 for Llama-derived anti-Human TGF-β1) or mouse latent TGF-1 (8 of 11 mAbs called LMT-1 to LMT-3 and LMT-5 to LMT-12 for Llama-derived anti-Mouse TGF-1). None of the activating mAbs were directed against GARP, and none were cross-reactive against human and mouse latent TGF-β1 (FIG. 1A). Results obtained with the TGF-β1-activating mAbs and a few non-activating mAbs in reporter assays are shown in FIG. 1B (top panel: LMT mAbs; bottom panel: LHT mAbs). They are representative of at least 3 independent experiments. Hatched bars in FIG. 1B highlight TGF-β1-activating mAbs. Dilutions of recombinant, active human TGF-β1 (rhTGF-1) were used as controls to monitor detection of TGF-β1 activity. Latent TGF-β1 activating mAbs triggered a signal equivalent to 40 to 320 μg/ml of rhTGF-β1. Serial dilutions of several TGF-1-activating mAbs were tested in the reporter assay to determine EC50s, which ranged from 0.2 to 2.1 nM (FIG. 1C).

### Example 2: Antibodies Against Mouse or Human Latent TGF-31 Activate TGF-31 when Used as Bivalent, Monospecific Full-Length mAbs or F(Ab′)2 Fragments, but not as Monovalent Fab Fragments

We tested the ability of 4 TGF-β1-activating LMT antibodies (namely, LMT-5, -7, -9 and -10) to activate latent TGF-β1 when used in various formats in the reporter assay described above (FIG. 2A). The 4 LMT activating antibodies activated TGF-31 when used as bivalent full length mAbs or F(ab′)2 fragments, whereas none activated TGF-β1 when used as monovalent Fab fragments (FIG. 2A). We concluded that bivalent binding to latent TGF-β1 is required for monospecific antibodies to mediate TGF-β1 activation.

### Example 3: TGF-β1-Activating Antibodies Activate Latent TGF-31 Whether or not Latent TGF-β1 is Presented by GARP on the Cell Surface

TGF-β1 activating mAbs were identified by screening in HEK-293 reporter cells that overexpress transmembrane protein GARP and latent TGF-β1, to maximize expression and presentation of latent TGF-β1 on the cell surface. Indeed, during latent TGF-β1 production and processing within cells, GARP binds to the LAP moiety of latent TGF-β1 via disulfide linkage, increases latent TGF-β1 production by acting as a chaperone, and finally presents and tethers latent TGF-β1 on the cell surface (Gauthy et al. PLoS One 2013; 8 (9): e76186; Lienart et al. Science 2018; 362 (6417): 952-956). We tested whether TGF-β1-activating mAbs could also activate latent TGF-β1 in the absence of GARP in a reporter assay in which HEK-293 cells were transfected with a plasmid encoding latent TGF-β1, with or without a plasmid encoding GARP (FIG. 2B). All 3 tested TGF-β1-activating mAbs LMT-7, -9 and -10 induced TGF-β1 activity well above background in reporter cells in the presence of GARP (FIG. 2B, left panel). Two of the 3 mAbs, namely LMT-7 and -10 also induced TGF-β1 activity above background in the absence of GARP, although to lower levels than in the presence of GARP (FIG. 2B, middle panel). Reduced activity likely results from lower levels of latent TGF-β1 tethered on the cell surface in the absence of GARP. None of the 3 mAbs induced TGF-β1 activity in reporter cells that had not been transfected to express latent TGF-β1 (FIG. 2B, right panel). This indicated that the TGF-β1-activating mAb preparations were not contaminated with active TGF-β1 that could have been co-purified with the mAbs. We confirmed that active TGF-β1 was not contaminating mAb preparations by ELISA (data not shown) and immunoblotting (FIG. 2C).

### Example 4: Unique Epitopes Bound by TGF-β1-Activating LMT and LHT mAbs

TGF-β1 activating mAbs bind either human or mouse TGF-β1, but not both (non-cross reactive mAbs). We used this observation to map the epitopes bound by human or mouse TGF-β1-activating mAbs, respectively. Sequence alignment of the human and mouse TGF-β1 proteins shows that most amino-acid differences are located between residues 87 and 278, a region of the LAP that is located outside the highly conserved so-called “latency lasso” (FIG. 3A). We constructed 4 plasmids encoding HA-tagged mouse-human-mouse chimeric TGF-1. In each chimera, amino-acids from one segment of mouse LAP were replaced by the amino-acids found in the corresponding segment of human LAP. The segments of mouse LAP replaced by the corresponding human LAP in the four plasmids comprised residues 87-135, 136-200, 201-222, or 223-278, respectively (FIG. 3B). We co-transfected plasmids encoding GARP and chimeric TGF-β1 in HEK-293T cells to express the chimeric forms of latent TGF-1 on the cell surface, and analyzed binding of TGF-β1-activating and non-activating mAbs to transfected cells by flow cytometry.

As expected, all LMT mAbs bound to cells expressing mouse TGF-β1, but not human TGF-β1 (FIG. 3B). Loss of binding of any given LMT mAb to a particular mouse-human-mouse chimera by comparison to mouse TGF-β1, allows to identify the region of mouse TGF-β1 that is required for binding by that particular mAb. We observed that 5 activating LMT mAbs (LMT-5, -6, -7, -10 and -11) required region 136-200 for binding (FIG. 3B). We could refine this binding requirement more precisely to residues 158-163 within region 136-200 (data not shown). Three other activating LMT mAbs (LMT-2, -9 and -12) required region 201-222 for binding (FIG. 3B). In contrast, non-activating LMT-1 mAb required both regions 136-200 and 201-222 for binding (LMT-1), whereas non-activating LMT-8 required none of the 4 regions tested in the chimeras (FIG. 3B).

As expected also, all LHT mAbs bound to cells expressing human TGF-β1, but not mouse TGF-31 (FIG. 3B). Here, gain of binding of any given LHT mAb to a particular mouse-human-mouse chimera by comparison to mouse TGF-β1 identifies the region required for binding by that particular mAb. We observed that 2 activating LHT mAbs (LHT-18 and -22) required region 201-222 for binding (i.e. the same region required by activating LMT-2, -9 and -12). Non-activating LHT-8 mAb also required region 201-222 for binding, whereas activating LHT-15 mAb required region 87-135.

Altogether, we observed that region 201-222, but no other region, was required for binding by 3 activating LMT and 2 activating LHT mAbs, highlighting a common binding requirement for activating mAbs directed against mouse or human TGF-β1, respectively. Two non-activating mAbs (LHT-8 shown in FIG. 3C and LHT-11, not shown) also required that region but no other for binding.

To further define the amino acids required for binding by activating mAbs in region 201-222, we constructed a series of plasmids, each encoding HA-tagged mouse or human TGF-β1 in which a single amino-acid was replaced by alanine (A). A series of 20 Ala-mutant plasmids covered the 20 positions of region 201-222 in mouse TGF-β1, another series of 20 plasmids covered the 20 positions in human TGF-β1 (FIG. 3C). To perform “Ala-scan” analyses, we co-transfected plasmids encoding mouse or human GARP and the single Ala-mutants of mouse or human TGF-β1 in HEK-293T cells, and analyzed binding by TGF-β1-activating and non-activating mAbs using flow cytometry (FIG. 3C).

Mouse TGF-β1-activating mAbs LMT-2 and LMT-12 lost the ability to bind to mouse TGF-β1 when L in position 208 (L208) or D in position 212 (D212) were mutated to A. Thus, activating LMT-2 and LMT-12 required L208 and D212, but no other single residue, for binding to mouse TGF-β1. No other LMT or LHT mAb binding to region 201-222, whether activating or non-activating, required L208 and D212 but no other amino-acid for binding. For example, non-activating LMT-1 mAb required L208, G211, D212 and F217, whereas non-activating LMT-8 mAb required none of the 20 single residues in region 201-222. Requirement for L208/D212 is unique to TGF-β1 activating mAbs LMT-2 and LMT-12, and is not observed with non-activating mAbs binding human or mouse TGF-β1.

Human TGF-β1-activating mAb LHT-22 lost the ability to bind to human TGF-β1 when G in position 212 (G212) or E in position 215 (E215) were mutated to A. Thus, LHT-22 required G212 and E215, but no other single residue, for binding to human TGF-β1. No other LMT or LHT mAb binding to region 201-222, whether activating or non-activating, required G212 and E215 but no other amino-acid for binding. Non-activating LHT-8 mAb required E213 and E215, whereas non-activating LHT-11 required R205 and R210. The requirement for G212/E215 is unique to activating mAb LHT-22, and is not observed with non-activating mAbs binding human or mouse TGF-β1.

Altogether, these observations revealed that binding requirements for L208/D212 in murine TGF-β1 or G212/E215 in human TGF-β1 are unique to TGF-β1-activating mAbs.

We also examined whether LMT-12 and LHT-22 lost the ability to activate single-A mutants in 293T reporter cells (FIG. 3D). As expected, the mAbs lost the ability to activate mutants to which they do not bind (FIG. 3C: L208A and D212A for LMT-12; G212A and E215A for LHT-22). But in addition, LHT-22 also lost the ability to activate 5 single-A mutants (W207A, L208A, G211A, 1214A, and F217A) to which it does binds as well as to WT TGF-β1. Thus, we identified 5 other TGF-β1 residues which are required for the activity of LHT-22.

Altogether, using chimeras and single A-mutants, we identified residues that are required for mAb binding and activity (FIG. 3B, C, D). To activate hTGF-β1, LHT-22 requires residues W207, L208, G211, G212, 1214, E215 and F217 in LAP. To activate mTGF-β1, LMT-12 requires residues L208 and D212 in LAP. LHT-22 and LMT-12 epitopes are very similar to one another and are not shared with non-activating mAbs. Interestingly, all TGF-β1 activating mAbs appear to bind loops close to the LAP dimerization interface (FIG. 2D). This interface was described to be critical for TGF-β1 latency, by keeping the LAP monomers welded together (Shi et al).

### Example 5: Treatment of Mice with TGF-β1-Activating LMT-10 and LMT-12 Increased Survival in a Model of Graft-Versus-Host Disease

Graft-versus-Host disease (GvHD) is a life-threatening side effect of allogeneic hematopoietic stem cell transplantation, due to a strong immune activity of donor-derived T cells against allogeneic recipient cells. Because of its potent immune-regulatory properties, TGF-β1 could reduce the activity of donor-derived T cells against allogeneic recipient antigens. We thus tested the therapeutic efficacy of TGF-β1-activating mAbs to treat GvHD in mice. We used a mouse model of GvHD in which splenocytes from C57BL/6 H-2^{b/b} mice are injected intraperitoneally (i.p.) in F1 (C57BL/6×DBA2) H-2^{b/d} recipients. Reactivity of donor T cells against allogenic H-2^{d} MHC molecules in F1 recipients leads to the development of GvHD, which can be scored by monitoring weight loss, hunched back posture, loss of mobility and ruffled fur, and can lead to death 10-21 days after donor cell transfer. We administered TGF-β1-activating mAbs or isotype controls (400 μg/mouse by weekly i.p. injections) to F1 H-2^{b/d} recipients, starting one day before the transfer of H-2^{b/b} splenocytes in three independent experiments (FIG. 4A).

Mice receiving TGF-β1-activating LMT-10 or LMT-12 had a significantly improved survival by comparison to mice receiving isotype control antibodies (FIG. 4B). Median survival was increased from 16 days in mice receiving the isotype control to 27 days in mice treated with LMT-10. More than 50% of mice treated with LMT-12 showed even long-term survival (>30 days), precluding estimation of median survival (FIG. 4B). GvHD scores in individual mice treated with LMT-10 or LMT-12 showed slower disease progression or reduced disease severity by comparison to isotype controls, or yet complete GvHD resolution (FIG. 4C).

### Example 6: LMT-12 Activated Latent TGF-β1 from a GARP-Independent Source in GvHD

As described in Example 4 and FIG. 2, TGF-β1-activating mAbs can activate latent TGF-β1 whether or not latent TGF-1 is presented by GARP on the cell surface. Notwithstanding this, when latent TGF-β1 is presented by GARP in HEK-293 cells, activation by TGF-β1-activating mAbs is completely inhibited in the presence of a blocking anti-GARP mAb. As shown in FIG. 5A, LHG-10, a blocking anti-human GARP mAb, inhibits human TGF-β1 activation by LHT-4, -15, -18, -20, -22 and -25 (left panel), and 58A2, a blocking anti-mouse GARP: latent TGF-β1 complex mAb, inhibits mouse TGF-β1 activation by LMT-12 (right panel). This observation provided an opportunity to measure whether TGF-β1-activating mAbs could still exert therapeutic activity in murine GvHD in the presence of blocking anti-mouse GARP antibody, indicating that TGF-β1-activating mAbs can activate latent TGF-β1 independently from latent TGF-β1 presentation by GARP-expressing cells in vivo.

We transferred F1 H-2^{b/d} recipient mice with H-2^{b/b} splenocytes as above, and treated recipient mice with TGF-β1-activating LMT-12 alone or in combination with blocking anti-GARP: latent TGF-β1 complex mAb 58A2 (FIG. 5B). As expected, 58A2 alone exerted no therapeutic effect, whereas LMT-12 alone exerted significant therapeutic effect in GvHD, increasing median survival, slowing disease progression and reducing disease severity by comparison to isotype control antibody. Interestingly, LMT-12 exerted the same therapeutic activity when it was administered alone or in combination with 58A2 (FIG. 5B). We thus concluded that TGF-β1-activating mAbs can exert therapeutic activity by activating latent TGF-1 produced by cells that do not express GARP in vivo. TGF-β1-activation by and therapeutic activity of activating mAbs does not depend on the context and are independent of GARP: TGF-β1 complexes.

### Example 7: Binding Activity of TGF-β1 Antibodies

The binding properties of the activating anti-human and anti-mouse latent TGFβ1 antibodies binding to region 201-222 were tested by surface plasmon resonance (Biacore 3000, GE Healthcare). A CM5 chip was immobilized with approximately 500RU human latent TGFβ1 and mouse latent TGFβ1 in sodium acetate buffer pH4.5 (GE Healthcare, #BR100350) using amine coupling and consuming HBS-EP buffer. The flow was set to 30 μl/min with the “kinject” injection mode and a regeneration injection of 10 mM glycine pH1.5. The on-rates (kₐ), off-rates (k_{d}) and affinities (K_{D}) were determined based on measurement of six concentrations per antibody: 100 nM, 50 nM, 25 nM, 12 nM, 6 nM and 3 nM. All clones tested were in hIgG1 backbone except for LHT-18, which had a ScFv-Fc hIgG1 backbone. All clones demonstrated binding to immobilized latent TGFβ1, with K_{D} values of 66 nM for LHT-18 and 2320 nM for LHT-22 binding human latent TGFβ1 and with K_{D} values ranging from 63 nM to 687 nM for clones binding mouse latent TGFβ1 (Table 4).

TABLE 4
Affinity determinations as determined by SPR

| | Affinity determination (SPR) | | | | | | | |
|---|---|---|---|---|---|---|---|---|
| | Human latent TGFβ1 | | | | Mouse latent TGFβ1 | | | |
| | ka | kd | Amplitude | KD | ka | kd | Amplitude | KD |
| Clones | (1/Ms) | (1/s) | R0 (RU) | (M) | (1/Ms) | (1/s) | R0 (RU) | (M) |
| LHT-22 | 1.42E+05 | 9.41E−05 | 303 | 6.63E−10 | NB | NB | NB | NB |
| (ScFv-Fc) | | | | | | | | |
| LHT-18 | 3.82E+05 | 8.86E−07 | 228 | 2.32E−12 | NB | NB | NB | NB |
| (hIgG1) | | | | | | | | |
| LMT-2 | NB | NB | NB | NB | 1.14E+06 | 7.18E−04 | 241 | 6.33E−10 |
| (hIgG1) | | | | | | | | |
| LMT-9 | NB | NB | NB | NB | 6.18E+05 | 4.65E−04 | 210 | 7.53E−10 |
| (hIgG1) | | | | | | | | |
| LMT-12 | NB | NB | NB | NB | 7.13E+05 | 4.90E−05 | 164 | 6.87E−11 |
| (hIgG1) | | | | | | | | |

NB = no binding

### Example 8: Specificity Determination Assays

The specificity of LHT-22 and LHT-18 to latent TGF-β1 compared to latent TGF-β2 and latent TGF-β3 was tested through an ELISA experiment for human latent TGF-β and through flow cytometry for mouse latent TGF-β, both through a single experiment.

An ELISA was performed to ensure that LHT-22 binds specifically to human latent TGF-β1 and not to human latent TGF-β2 and human latent TGF-β3. Briefly, a 96-well Maxisorp plate was coated with human latent TGF-β1, TGF-β2 or TGF-β3. Three different concentrations of LHT-22 hIgG1 were then incubated, and bound antibody was detected with an anti-hIgG-HRP antibody. Antibodies having an OD value<0.1 were defined as non-binding. A negative control sample (Motavizumab) was included as well. All concentrations tested (4, 1 and 0.25 μg/mL) for LHT-22 showed binding to human latent TGF-β1 and not to human latent TGF-β2 and latent TGF-β3 (Table 5).

Flow cytometry was used to ensure that LMT-12 binds specifically to mouse latent TGF-β1 and not to mouse latent TGF-β2 and mouse latent TGF-β3. Briefly, 293T cells were transfected with plasmids encoding HA-tagged TGF-β1, β2, or β3, with a plasmid encoding GARP. They were then stained with the indicated concentrations of LMT-12 mIgG1 or anti-HA mIgG1 antibody and thereafter with goat anti-mouse coupled to FITC (GAM-FITC) as secondary reagent. Using flow cytometry, histograms were gated on live cells as determined with a viability dye (Bioscience, cat. 65-0865-14). All concentrations tested for LMT-12 showed binding to mouse latent TGF-β1 and not to mouse latent TGF-β2 and latent TGF-β3 (FIG. 6).

TABLE 5
Specificity testing of LHT-22 for human latent
TGFβ1 through ELISA (OD 450 nm values)

| | OD value | OD value | OD value |
|---|---|---|---|
| | human latent | human latent | human latent |
| Clone | TGF-β1 | TGF-β2 | TGF-β3 |
| LHT-22 - 4 μg/mL | 1.84 | 0.071 | 0.045 |
| LHT-22 - 1 μg/mL | 1.827 | 0.066 | 0.045 |
| LHT-22 - 0.25 μg/mL | 1.757 | 0.046 | 0.028 |
| Isotype control - 4 μg/mL | 0.004 | 0.007 | 0.001 |

TABLE 6
Heavy chain CDR sequences of Abs binding to latent TGF-β1

| | | SEQ ID | | SEQ ID | | SEQ ID |
|---|---|---|---|---|---|---|
| Fab clone | CDR1 | NO. | CDR2 | NO. | CDR3 | NO. |
| HUMAN | | | | | | |
| LHT-22 (15A2) | DYTMN | 9 | AIRWNGVTTYYAESMKG | 10 | GGSIDLTYGMDY | 11 |
| LHT-18 (14E7) | HYWMY | 12 | AINTGGGITYYTDSVKG | 13 | SRYGWVITTTYFGGVDY | 14 |
| LHT-15 (13E7) | SYWMD | 55 | RITNSGGTAFYTDSVEG | 56 | YITRAQLMGAWDY | 57 |
| MOUSE | | | | | | |
| LMT-2 (6D12) | NYGVH | 15 | HISAAGISRYYADAVKG | 16 | DRIRSSYEYDS | 17 |
| LMT-9 (20G3) | RSGMA | 18 | AITPGGTATYYANSVKG | 19 | GLVSSGLNGMDY | 20 |
| LMT-12 (14D7) | SYYMY | 21 | GTSIGGGSTWYADSVKG | 22 | DLNSRSYKGMGD | 23 |
| LMT-10 (20G4) | NYYMT | 47 | SIYSTGSHTYYADSVKG | 48 | NIYTYYTAHYAGMDY | 49 |

TABLE 7
Light chain CDR sequences of Abs binding to latent TGF-1

| | | SEQ ID | | SEQ ID | | SEQ ID |
|---|---|---|---|---|---|---|
| Fab clone | CDR1 | NO. | CDR2 | NO. | CDR3 | NO. |
| HUMAN | | | | | | |
| LHT-22 (15A2) | GLSSGSVTRNNYPD | 24 | NTVARHS | 25 | ALYMYTGSNNGRV | 26 |
| LHT-18 (14E7) | QGGNFGSYYAS | 27 | GNSNRPS | 28 | LSYESSGYAV | 29 |
| LHT-15 (13E7) | KASQSLVHTDGKTY | 58 | QVSNRAS | 59 | AQSTYYPYD | 60 |
| | LS | | | | | |
| MOUSE | | | | | | |
| LMT-2 (6D12) | TGSSSNIGGGYYLS | 30 | NANNRAS | 31 | GCYDSSLSTGYV | 32 |
| LMT-9 (20G3) | TGSSSNIGGGYYLS | 30 | NTNNRAS | 33 | GCYDDSLNTYV | 34 |
| LMT-12 (14D7) | TGSSSNIGGGYYLN | 35 | NANNRAS | 31 | GCYDSSLSSVV | 36 |
| LMT-10 (20G4) | QGGSLGSYDAH | 50 | DDNSRPS | 51 | HSYDSSANAPV | 52 |

TABLE 8
VH and VL sequences of Abs binding to latent TGF-1

| | | SEQ | | SEQ |
|---|---|---|---|---|
| Fab | | ID | | ID |
| clone | VH | NO. | VL | NO. |
| HUMAN | | | | |
| LHT-22 | ELQLVESGGGLVQPGGSLRLSCAASGFTFDDY | 37 | QAVVTQEPSLSVSPGGTVTITCGLSSGSVTRNNYPDW | 38 |
| (15A2) | TMNWVRQAPGKGLEWVSAIRWNGVTTYYAESM | | YQQTPGQAPRLLLYNTVARHSGVPSRFSGSISGNKAA | |
| | KGRFTVSRDNGNNTLYLQMNSLKAEDTAVYY | | LTITGAQPEDEAGYYCALYMYTGSNNGRVFGGGTLLT | |
| | CAKGGSIDLTYGMDYWGKGTLVTVSS | | VL | |
| LHT-18 | QVQLVESGGGLVQPGGSLRLSCAASGFTFSHY | 39 | SSALTQPSAVSVSLGQTARITCQGGNFGSYYASWYQQ | 40 |
| (14E7) | WMYWVRQAPGKGLEWVSAINTGGGITYYTDSV | | KPGQAPVQVIYGNSNRPSGIPERFSGSSSGDTATLTI | |
| | KGRFTVSRDNAKNTLYLQMNSLKSEDTAVYYC | | SGAQAEDEADYYCLSYESSGYAVFGGGTHLTVL | |
| | AKSRYGVVITTTYFGGVDYWGKGTLVTVSS | | | |
| LHT-15 | EVQLVESGGDLVQPGGSLRLSCAASGFTFSS | 61 | ATMLTQSPGSLSVVPGESASISCKASQSLVHTDGK | 62 |
| (13E7) | YWMDWVRQAPGKGLEWVSRITNSGGTAFYTD | | TYLSWLLQKPGQRPQLLIYQVSNRASGVPDRFTGS | |
| | SVEGRFTISRDNAKNTLYLQMNSLKSEDTAV | | GSGTDFTLKISGVKAEDAGVYYCAQSTYYPYDFGS | |
| | YYCLRYITRAQLMGAWDYWGQGTQVTVSS | | GTRLEIR | |
| MOUSE | | | | |
| LMT-2 | EVQLVESGGGLVQPGGSLRLSCAASGFTFSNY | 41 | LPVLTQLSSMSGSPGQTVTITCTGSSSNIGGGYYLSW | 42 |
| (6D12) | GVHWVRQAPGKGLEWVSHISAAGISRYYADAV | | YQQLPGTAPKLLIYNANNRASGVPNRFSGSKSGNLAS | |
| | KGRFTMSRDNGKNTLYLQMNGLKSEDTAVYYC | | LTITGLQAEDEADYYCGCYDSSLSTGYVFGGGTKLTV | |
| | AKDRIRSSYEYDSWGQGTQVTVSS | | L | |
| LMT-9 | LQLVESGGGLVQPGGSLRLSCAASGFTFSRSG | 43 | QSVLTQLSSMSGSPGQTVTITCTGSSSNIGGGYYLSW | 44 |
| (20G3) | MAWVRQAPGKGLEWVSAITPGGTATYYANSVK | | YQQLPGTAPKLLIYNTNNRASGVPNRFSGSKTGSLAS | |
| | GRVTMSRDNAKNTLYLQMNSLKSEDTAVYYCA | | LTITGLQAEDEADYYCGCYDDSLNTYVFGGGTKLTVL | |
| | KGLVSSGLNGMDYWGKGTLVTVSS | | | |
| LMT-12 | EVQLVESGGGLVQPGGSLRLSCAASGFTFSSY | 45 | QPALTQLSSMSGSPGQTVTITCTGSSSNIGGGYYLNW | 46 |
| (14D7) | YMYWVRQAPGKGLEWVSGTSIGGGSTWYADSV | | YQQLPGAAPKLVIYNANNRASGVPDRFSGSKSGNLAS | |
| | KGRFTISRDNAKNTLYLQMNNLKSEDTAVYYC | | LTITGLQAEDEAVYFCGCYDSSLSSVVFGGGTHLTVL | |
| | AKDLNSRSYKGMGDWGQGTQVTVSS | | | |
| LMT-10 | QVQLVESGGGLVQPGGSLTLSCAASGFTFSNY | 53 | QSALTQPSTVSVSLGQMARITCQGGSLGSYDAHWYQ | 54 |
| (20G4) | YMTWVRQAPGKGLEWVSSIYSTGSHTYYADSV | | QKPGQAPVLVIHDDNSRPSGIPERFSGSRSGGTATL | |
| | KGRFTISRDNAKDTLYLQMNDLKSEDTAVYYC | | TISGAQADDEGVYFCHSYDSSANAPVFGGGTELTVL | |
| | AKNIYTYYTAHYAGMDYWGKGTLVTVSS | | | |

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

### INCORPORATION BY REFERENCE

Various publications are cited in the foregoing description and throughout the following examples, each of which is incorporated by reference herein in its entirety.

1-40. (canceled)

41. An antibody, or an antigen binding fragment thereof, which binds to latent TGF-β1, wherein the antibody or antigen binding fragment thereof activates TGF-β1.

42. The antibody or antigen binding fragment of claim 41, which activates TGF-β1 by releasing the mature TGF-β1 cytokine from the inhibition mediated by the latency associated peptide (LAP).

43. The antibody or antigen binding fragment of claim 41, which binds to an epitope comprising one or more amino acids from the region of human or murine latent TGF-β1 consisting of amino acids 87-222, wherein human latent TGF-β1 is represented by SEQ ID NO: 1 and murine latent TGF-β1 is represented by SEQ ID NO: 5.

44. The antibody or antigen binding fragment of claim 41, which binds to an epitope comprising one or more amino acids from the region of human or murine latent TGF-β1 consisting of amino acids 87-135, wherein human latent TGF-β1 is represented by SEQ ID NO: 1 and murine latent TGF-β1 is represented by SEQ ID NO: 5.

45. The antibody or antigen binding fragment of claim 41, which binds to an epitope comprising one or more amino acids from the region of human or murine latent TGF-β1 consisting of amino acids 136-200, wherein human latent TGF-β1 is represented by SEQ ID NO: 1 and murine latent TGF-β1 is represented by SEQ ID NO: 5.

46. The antibody or antigen binding fragment of claim 41, which binds to an epitope comprising one or more amino acids from the region of human or murine latent TGF-β1 consisting of amino acids 201-222, wherein human latent TGF-β1 is represented by SEQ ID NO: 1 and murine latent TGF-β1 is represented by SEQ ID NO: 5.

47. The antibody or antigen binding fragment of claim 43, which binds to human latent TGF-β1.

48. The antibody or antigen binding fragment of claim 47, which binds to an epitope comprising amino acid residues E119, K123, S138, R141, S209, R210, G212, E213, I214, and/or E215 of human latent TGF-β1, wherein human latent TGF-β1 is represented by SEQ ID NO: 1.

49. The antibody or antigen binding fragment of claim 43, which binds to murine latent TGF-β1.

50. The antibody or antigen binding fragment of claim 49, which binds to an epitope comprising amino acid residues S138, R141, P145, E146, S175, L208, N209 and/or D212 of murine latent TGF-β1, wherein murine latent TGF-β1 is represented by SEQ ID NO: 5.

51. The antibody or antigen binding fragment of claim 50, which binds to an epitope comprising amino acid residues S138, R141, P145, E146, S175, L208, N209 and/or D212 of murine latent TGF-β1, wherein the epitope does not comprise another amino acid residue from within the region of murine latent TGF-β1 consisting of amino acids 135-222, wherein murine latent TGF-β1 is represented by SEQ ID NO: 5.

52. The antibody or antigen binding fragment of claim 41, which activates TGF-β1 when latent TGF-β1 is complexed with GARP.

53. The antibody or antigen binding fragment of claim 41, which activates TGF-β1 when latent TGF-β1 is bound to the surface of one or more cells types selected from: regulatory T cells (Tregs); megakaryocytes; platelets; B lymphocytes; endothelial cells; fibroblasts; mesenchymal cells; and hepatic stellate cells.

54. The antibody or antigen binding fragment of claim 41, which activates latent TGF-β1 associated with one or more cell types selected from: myeloid cells; lymphoid cells; hematopoietic cells; and cancer cells at a pre-malignant stage.

55. The antibody or antigen binding fragment of claim 41, wherein the antibody or antigen binding fragment is bivalent.

56. The antibody or antigen binding fragment of claim 55, wherein the antibody is an IgG.

57. The antibody or antigen binding fragment of claim 55, wherein the antibody or antigen binding fragment comprises the CH1 domain, hinge region, CH2 domain and/or CH3 domain of a human IgG.

58. The antigen binding fragment of claim 41, wherein the antigen binding fragment is selected from the group consisting of: an antibody light chain variable domain (VL); an antibody heavy chain variable domain (VH or VHH); a single chain antibody (scFv); a (scFv)2 fragment, a F(ab′)2 fragment; a Fab fragment; an Fd fragment; an Fv fragment; a one-armed (monovalent) antibody; diabodies; triabodies; and tetrabodies.

59. The antibody or antigen binding fragment of claim 41, wherein the antibody or antigen binding fragment comprises a variable heavy chain domain (VH) and a variable light chain domain (VL) wherein the VH and VL domains comprise the CDR sequences selected from the group consisting of:
(i) HCDR3 comprising or consisting of SEQ ID NO: 11; HCDR2 comprising or consisting of SEQ ID NO: 10; HCDR1 comprising or consisting of SEQ ID NO: 9; LCDR3 comprising or consisting of SEQ ID NO: 26; LCDR2 comprising or consisting of SEQ ID NO: 25; LCDR1 comprising or consisting of SEQ ID NO: 24;
(ii) HCDR3 comprising or consisting of SEQ ID NO: 14; HCDR2 comprising or consisting of SEQ ID NO: 13; HCDR1 comprising or consisting of SEQ ID NO: 12; LCDR3 comprising or consisting of SEQ ID NO: 29; LCDR2 comprising or consisting of SEQ ID NO: 28; LCDR1 comprising or consisting of SEQ ID NO: 27; and
(iii) HCDR comprising or consisting of SEQ ID NO: 57; HCDR2 comprising or consisting of SEQ ID NO: 56; HCDR1 comprising or consisting of SEQ ID NO: 55; LCDR3 comprising or consisting of SEQ ID NO: 60; LCDR2 comprising or consisting of SEQ ID NO: 59; LCDR1 comprising or consisting of SEQ ID NO: 58;
or any CDR having an amino acid sequence that shares at least about 90% of identity with SEQ ID NOs: 9-14, 24-29, 55-60.

60. The antibody or antigen binding fragment of claim 49, wherein the antibody or antigen binding fragment comprises a combination of a heavy chain variable domain (VH) and a light chain variable domain (VL) selected from the group consisting of:
(i) a VH comprising the amino acid sequence of SEQ ID NO: 37 or an amino acid sequence at least 90%, 95%, 97%, 98% or 99% identical thereto and a VL comprising the amino acid sequence of SEQ ID NO: 38 or an amino acid sequence at least 90%, 95%, 97%, 98% or 99% identical thereto;
(ii) a VH comprising the amino acid sequence of SEQ ID NO: 39 or an amino acid sequence at least 90%, 95%, 97%, 98% or 99% identical thereto and a VL comprising the amino acid sequence of SEQ ID NO: 40 or an amino acid sequence at least 90%, 95%, 97%, 98% or 99% identical thereto; and
(iii) a VH comprising the amino acid sequence of SEQ ID NO: 61 or an amino acid sequence at least 90%, 95%, 97%, 98% or 99% identical thereto and a VL comprising the amino acid sequence of SEQ ID NO: 61 or an amino acid sequence at least 90%, 95%, 97%, 98% or 99% identical thereto.

61. The antibody or antigen binding fragment of claim 41, wherein the antibody or antigen binding fragment comprises a variable heavy chain domain (VH) and a variable light chain domain (VL) wherein the VH and VL domains comprise the CDR sequences selected from the group consisting of:
(i) HCDR3 comprising or consisting of SEQ ID NO: 17; HCDR2 comprising or consisting of SEQ ID NO: 16; HCDR1 comprising or consisting of SEQ ID NO: 15; LCDR3 comprising or consisting of SEQ ID NO: 32; LCDR2 comprising or consisting of SEQ ID NO: 31; LCDR1 comprising or consisting of SEQ ID NO: 30;
(ii) HCDR3 comprising or consisting of SEQ ID NO: 20; HCDR2 comprising or consisting of SEQ ID NO: 19; HCDR1 comprising or consisting of SEQ ID NO: 18; LCDR3 comprising or consisting of SEQ ID NO: 34; LCDR2 comprising or consisting of SEQ ID NO: 33; LCDR1 comprising or consisting of SEQ ID NO: 30; and
(iii) HCDR3 comprising or consisting of SEQ ID NO: 23; HCDR2 comprising or consisting of SEQ ID NO: 22; HCDR1 comprising or consisting of SEQ ID NO: 21; LCDR3 comprising or consisting of SEQ ID NO: 36; LCDR2 comprising or consisting of SEQ ID NO: 31; LCDR1 comprising or consisting of SEQ ID NO: 35;
or any CDR having an amino acid sequence that shares at least about 90% of identity with SEQ ID NOs: 15-23, 30-36.

62. The antibody or antigen binding fragment of claim 61, wherein the antibody or antigen binding fragment comprises a combination of a heavy chain variable domain (VH) and a light chain variable domain (VL) selected from the group consisting of:
(i) a VH comprising the amino acid sequence of SEQ ID NO: 41 or an amino acid sequence at least 90%, 95%, 97%, 98% or 99% identical thereto and a VL comprising the amino acid sequence of SEQ ID NO: 42 or an amino acid sequence at least 90%, 95%, 97%, 98% or 99% identical thereto;
(ii) a VH comprising the amino acid sequence of SEQ ID NO: 43 or an amino acid sequence at least 90%, 95%, 97%, 98% or 99% identical thereto and a VL comprising the amino acid sequence of SEQ ID NO: 44 or an amino acid sequence at least 90%, 95%, 97%, 98% or 99% identical thereto; and
(iii) a VH comprising the amino acid sequence of SEQ ID NO: 45 or an amino acid sequence at least 90%, 95%, 97%, 98% or 99% identical thereto and a VL comprising the amino acid sequence of SEQ ID NO: 46 or an amino acid sequence at least 90%, 95%, 97%, 98% or 99% identical thereto.

63. An antibody or antigen binding fragment thereof that binds to latent TGF-β1, wherein the antibody or antigen binding fragment comprises a variable heavy chain domain (VH) and a variable light chain domain (VL) wherein the VH and VL domains comprise the CDR sequences selected from the group consisting of:
(i) HCDR3 comprising or consisting of SEQ ID NO: 11; HCDR2 comprising or consisting of SEQ ID NO: 10; HCDR1 comprising or consisting of SEQ ID NO: 9; LCDR3 comprising or consisting of SEQ ID NO: 26; LCDR2 comprising or consisting of SEQ ID NO: 25; LCDR1 comprising or consisting of SEQ ID NO: 24;
(ii) HCDR3 comprising or consisting of SEQ ID NO: 14; HCDR2 comprising or consisting of SEQ ID NO: 13; HCDR1 comprising or consisting of SEQ ID NO: 12; LCDR3 comprising or consisting of SEQ ID NO: 29; LCDR2 comprising or consisting of SEQ ID NO: 28; LCDR1 comprising or consisting of SEQ ID NO: 27; and
(iii) HCDR3 comprising or consisting of SEQ ID NO: 57; HCDR2 comprising or consisting of SEQ ID NO: 56; HCDR1 comprising or consisting of SEQ ID NO: 55; LCDR3 comprising or consisting of SEQ ID NO: 60; LCDR2 comprising or consisting of SEQ ID NO: 59; LCDR1 comprising or consisting of SEQ ID NO: 58;
or any CDR having an amino acid sequence that shares at least about 90% of identity with SEQ ID NOs: 9-14, 24-29, 55-60.

64. The antibody or antigen binding fragment thereof of claim 63, wherein the antibody or antigen binding fragment comprises a combination of a heavy chain variable domain (VH) and a light chain variable domain (VL) selected from the group consisting of:
(i) a VH comprising the amino acid sequence of SEQ ID NO: 37 or an amino acid sequence at least 90%, 95%, 97%, 98% or 99% identical thereto and a VL comprising the amino acid sequence of SEQ ID NO: 38 or an amino acid sequence at least 90%, 95%, 97%, 98% or 99% identical thereto;
(ii) a VH comprising the amino acid sequence of SEQ ID NO: 39 or an amino acid sequence at least 90%, 95%, 97%, 98% or 99% identical thereto and a VL comprising the amino acid sequence of SEQ ID NO: 40 or an amino acid sequence at least 90%, 95%, 97%, 98% or 99% identical thereto; and
(iii) a VH comprising the amino acid sequence of SEQ ID NO: 61 or an amino acid sequence at least 90%, 95%, 97%, 98% or 99% identical thereto and a VL comprising the amino acid sequence of SEQ ID NO: 62 or an amino acid sequence at least 90%, 95%, 97%, 98% or 99% identical thereto.

65. An antibody or antigen binding fragment thereof which binds to latent TGF-β1, wherein the antibody or antigen binding fragment comprises a variable heavy chain domain (VH) and a variable light chain domain (VL) wherein the VH and VL domains comprise the CDR sequences selected from the group consisting of:
(i) HCDR3 comprising or consisting of SEQ ID NO: 17; HCDR2 comprising or consisting of SEQ ID NO: 16; HCDR1 comprising or consisting of SEQ ID NO: 15; LCDR3 comprising or consisting of SEQ ID NO: 32; LCDR2 comprising or consisting of SEQ ID NO: 31; LCDR1 comprising or consisting of SEQ ID NO: 30;
(ii) HCDR3 comprising or consisting of SEQ ID NO: 20; HCDR2 comprising or consisting of SEQ ID NO: 19; HCDR1 comprising or consisting of SEQ ID NO: 18; LCDR3 comprising or consisting of SEQ ID NO: 34; LCDR2 comprising or consisting of SEQ ID NO: 33; LCDR1 comprising or consisting of SEQ ID NO: 30; and
(iii) HCDR3 comprising or consisting of SEQ ID NO: 23; HCDR2 comprising or consisting of SEQ ID NO: 22; HCDR1 comprising or consisting of SEQ ID NO: 21; LCDR3 comprising or consisting of SEQ ID NO: 36; LCDR2 comprising or consisting of SEQ ID NO: 31; LCDR1 comprising or consisting of SEQ ID NO: 35;
or any CDR having an amino acid sequence that shares at least about 90% of identity with SEQ ID NOs: 15-23, 30-36.

66. The antibody or antigen binding fragment thereof of claim 65, wherein the antibody or antigen binding fragment comprises a combination of a heavy chain variable domain (VH) and a light chain variable domain (VL) selected from the group consisting of:
(i) a VH comprising the amino acid sequence of SEQ ID NO: 41 or an amino acid sequence at least 90%, 95%, 97%, 98% or 99% identical thereto and a VL comprising the amino acid sequence of SEQ ID NO: 42 or an amino acid sequence at least 90%, 95%, 97%, 98% or 99% identical thereto;
(ii) a VH comprising the amino acid sequence of SEQ ID NO: 43 or an amino acid sequence at least 90%, 95%, 97%, 98% or 99% identical thereto and a VL comprising the amino acid sequence of SEQ ID NO: 44 or an amino acid sequence at least 90%, 95%, 97%, 98% or 99% identical thereto; and
(iii) a VH comprising the amino acid sequence of SEQ ID NO: 45 or an amino acid sequence at least 90%, 95%, 97%, 98% or 99% identical thereto and a VL comprising the amino acid sequence of SEQ ID NO: 46 or an amino acid sequence at least 90%, 95%, 97%, 98% or 99% identical thereto.

67. An antibody or antigen binding fragment thereof, which binds to the same epitope as the antibody of claim 63.

68. An isolated nucleic acid encoding the antibody or antigen binding fragment of claim 41, or a VH, VHH or VL domain thereof.

69. An expression vector comprising the nucleic acid of claim 68 operably linked to regulatory sequences, which permit expression of the antibody, antigen binding fragment, VH domain, VHH domain or VL domain in a host cell or cell-free expression system.

70. A host cell or cell-free expression system containing the expression vector of claim 69.

71. A process of producing a recombinant antibody or antigen binding fragment thereof which comprises culturing the host cell or cell free expression system of claim 70 under conditions which permit expression of the antibody or antigen binding fragment and recovering the expressed antibody or antigen binding fragment.

72. A pharmaceutical composition comprising an antibody or antigen binding fragment according to claim 41 and at least one pharmaceutically acceptable carrier or excipient.

73. A method comprising administering to a subject in need thereof, an antibody or antigen binding fragment according to claim 41 or a pharmaceutical composition comprising said antibody or antigen binding fragment and at least one pharmaceutically acceptable carrier or excipient.

74. A method of suppressing a pathological immune response in a subject in need thereof, wherein the method comprises administering to the subject a therapeutically effective amount of an antibody or antigen binding fragment according to claim 41.

75. A method of preventing or treating an autoimmune disease and/or an allo-immune disease in a subject in need thereof, wherein the method comprises administering to the subject a therapeutically effective amount of an antibody or antigen binding fragment according to claim 41, or a pharmaceutical composition comprising said antibody or antigen binding fragment and at least one pharmaceutically acceptable carrier or excipient.

76. The method of claim 75, wherein the disease is selected from the group consisting of: Inflammatory Bowel Disease (IBD), multiple sclerosis (MS), graft-versus-host disease (GVHD), allograft rejection, antibody-mediated allograft rejection (AMR), allogenic islet graft rejection, alopecia areata, ankylosing spondylitis, antiphospholipid syndrome (APS), autoimmune Addison's disease, Alzheimer's disease, antineutrophil cytoplasmic autoantibodies (ANCA), ANCA vasculitis, autoimmune diseases of the adrenal gland, autoimmune encephalitis, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopeniaurpura or idiopathic thrombocytopenia purpura or immune mediated thrombocytopenia), autoimmune urticaria, Behcet's disease, bullous pemphigoid (BP), cardiomyopathy, Castleman's syndrome, celiac spruce-dermatitis, chronic fatigue immune dysfunction syndrome, chronic inflammatory demyelinating polyneuropathy (CIDP), Churg-Strauss syndrome, cicatrical pemphigoid, CREST syndrome, cold agglutinin disease, COVID-19 mediated postural orthostatic tachycardia syndrome (POTS), Crohn's disease, delayed graft function after kidney transplant, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barré syndrome (GBS), Goodpasture's syndrome, Hashimoto's thyroiditis, hemophilia A, hemolytic disease of the fetus and newborn (HDFN), idiopathic membranous neuropathy, idiopathic pulmonary fibrosis, IgA neuropathy, IgM polyneuropathies, juvenile arthritis, Kawasaki's disease, lichen plantus, lichen sclerosus, lupus erthematosis, systemic lupus erythematosus (SLE), lupus nephritis, membranous neuropathy, membranous nephropathy, Méniere's disease, mixed connective tissue disease, mucous membrane pemphigoid, graft type 1 diabetes mellitus, multifocal motor neuropathy (MMN), myelin oligodendrocyte glycoprotein antibody-associated disease (MOGAD), myasthenia gravis (MG), generalized myasthenia gravis (gMG), ocular myasthenia gravis (OMG), myositis, neuromyelitis optica (NMO), paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Reynauld's phenomenon, Reiter's syndrome, rheumatoid arthritis (RA), sarcoidosis, scleroderma, Sjögren's syndrome, solid organ transplant rejection, stiff-man syndrome, takayasu arteritis, toxic epidermal necrolysis (TEN), Stevens Johnson syndrome (SJS), temporal arteristis/giant cell arteritis, thrombotic thrombocytopenia purpura, thyroid eye disease, ulcerative colitis, uveitis, warm autoimmune hemolytic anemia (wAIHA), dermatitis herpetiformis vasculitis, anti-neutrophil cytoplasmic antibody-associated vasculitides, vitiligo, and Wegner's granulomatosis.

77. A method of preventing or treating a neoplastic disease in a subject in need thereof, wherein the method comprises administering to the subject a therapeutically effective amount of an antibody or antigen binding fragment according to claim 41, a pharmaceutical composition comprising said antibody or antigen binding fragment and at least one pharmaceutically acceptable carrier or excipient.

78. A method for treating an autoimmune disease and/or an allo-immune disease in a subject in need thereof, wherein the method comprises administering to the subject an antibody or antigen binding fragment according to claim 41 or a pharmaceutical composition comprising said antibody or antigen binding fragment and at least one pharmaceutically acceptable carrier or excipient.

79. A method for treating a neoplastic disease in a subject in need thereof, wherein the method comprises administering to the subject an antibody or antigen binding fragment according to claim 41 or a pharmaceutical composition comprising said antibody or antigen binding fragment and at least one pharmaceutically acceptable carrier or excipient.

80. A kit comprising an antibody or antigen binding fragment according to claim 41 or a pharmaceutical composition comprising said antibody or antigen binding fragment and at least one pharmaceutically acceptable carrier or excipient.

81. An antibody or antigen binding fragment thereof, which binds to the same epitope as the antibody of claim 65.

82. An isolated nucleic acid encoding the antibody or antigen binding fragment of claim 63, or a VH, VHH or VL domain thereof.

83. An isolated nucleic acid encoding the antibody or antigen binding fragment of claim 65, or a VH, VHH or VL domain thereof.

84. An isolated nucleic acid encoding the antibody or antigen binding fragment of claim 67, or a VH, VHH or VL domain thereof.

85. An isolated nucleic acid encoding the antibody or antigen binding fragment of claim 81, or a VH, VHH or VL domain thereof.

86. An expression vector comprising the nucleic acid of claim 82 operably linked to regulatory sequences, which permit expression of the antibody, antigen binding fragment, VH domain, VHH domain or VL domain in a host cell or cell-free expression system.

87. An expression vector comprising the nucleic acid of claim 83 operably linked to regulatory sequences, which permit expression of the antibody, antigen binding fragment, VH domain, VHH domain or VL domain in a host cell or cell-free expression system.

88. An expression vector comprising the nucleic acid of claim 84 operably linked to regulatory sequences, which permit expression of the antibody, antigen binding fragment, VH domain, VHH domain or VL domain in a host cell or cell-free expression system.

89. An expression vector comprising the nucleic acid of claim 85 operably linked to regulatory sequences, which permit expression of the antibody, antigen binding fragment, VH domain, VHH domain or VL domain in a host cell or cell-free expression system.

90. A host cell or cell-free expression system containing the expression vector of claim 86.

91. A host cell or cell-free expression system containing the expression vector of claim 87.

92. A host cell or cell-free expression system containing the expression vector of claim 88.

93. A host cell or cell-free expression system containing the expression vector of claim 89.

94. A process of producing a recombinant antibody or antigen binding fragment thereof which comprises culturing the host cell or cell free expression system of claim 90 under conditions which permit expression of the antibody or antigen binding fragment and recovering the expressed antibody or antigen binding fragment.

95. A process of producing a recombinant antibody or antigen binding fragment thereof which comprises culturing the host cell or cell free expression system of claim 91 under conditions which permit expression of the antibody or antigen binding fragment and recovering the expressed antibody or antigen binding fragment.

96. A process of producing a recombinant antibody or antigen binding fragment thereof which comprises culturing the host cell or cell free expression system of claim 92 under conditions which permit expression of the antibody or antigen binding fragment and recovering the expressed antibody or antigen binding fragment.

97. A process of producing a recombinant antibody or antigen binding fragment thereof which comprises culturing the host cell or cell free expression system of claim 93 under conditions which permit expression of the antibody or antigen binding fragment and recovering the expressed antibody or antigen binding fragment.

98. A pharmaceutical composition comprising an antibody or antigen binding fragment according to claim 63 and at least one pharmaceutically acceptable carrier or excipient.

99. A pharmaceutical composition comprising an antibody or antigen binding fragment according to claim 65 and at least one pharmaceutically acceptable carrier or excipient.

100. A pharmaceutical composition comprising an antibody or antigen binding fragment according to claim 67 and at least one pharmaceutically acceptable carrier or excipient.

101. A pharmaceutical composition comprising an antibody or antigen binding fragment according to claim 81 and at least one pharmaceutically acceptable carrier or excipient.

102. A method comprising administering to a subject in need thereof an antibody or antigen binding fragment according to claim 63 or a pharmaceutical composition comprising said antibody or antigen binding fragment and at least one pharmaceutically acceptable carrier or excipient.

103. A method comprising administering to a subject in need thereof an antibody or antigen binding fragment according to claim 65 or a pharmaceutical composition comprising said antibody or antigen binding fragment and at least one pharmaceutically acceptable carrier or excipient.

104. A method comprising administering to a subject in need thereof an antibody or antigen binding fragment according to claim 67 or a pharmaceutical composition comprising said antibody or antigen binding fragment and at least one pharmaceutically acceptable carrier or excipient.

105. A method comprising administering to a subject in need thereof an antibody or antigen binding fragment according to claim 61 or a pharmaceutical composition comprising said antibody or antigen binding fragment and at least one pharmaceutically acceptable carrier or excipient.

106. A kit comprising an antibody or antigen binding fragment according to claim 63 or a pharmaceutical composition comprising said antibody or antigen binding fragment and at least one pharmaceutically acceptable carrier or excipient.

107. A kit comprising an antibody or antigen binding fragment according to claim 65 or a pharmaceutical composition comprising said antibody or antigen binding fragment and at least one pharmaceutically acceptable carrier or excipient.

108. A kit comprising an antibody or antigen binding fragment according to claim 67 or a pharmaceutical composition comprising said antibody or antigen binding fragment and at least one pharmaceutically acceptable carrier or excipient.

109. A kit comprising an antibody or antigen binding fragment according to claim 81 or a pharmaceutical composition comprising said antibody or antigen binding fragment and at least one pharmaceutically acceptable carrier or excipient.


SEQUENCE LISTING (87 sequences)

SEQ ID NO: 1 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 29 aa, Homo sapiens
MPPSGLRLLPLLLPLLWLLVLTPGRPAAG

SEQ ID NO: 3 - protein, 249 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 4 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 5 - protein, 390 aa, Mus musculus
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 6 - protein, 29 aa, Mus musculus
MPPSGLRLLPLLLPLPWLLVLTPGRPAAG

SEQ ID NO: 7 - protein, 249 aa, Mus musculus
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 8 - protein, 112 aa, Mus musculus
ALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 9 - protein, 5 aa, Synthetic construct
DYTMN

SEQ ID NO: 10 - protein, 17 aa, Synthetic construct
AIRWNGVTTYYAESMKG

SEQ ID NO: 11 - protein, 12 aa, Synthetic construct
GGSIDLTYGMDY

SEQ ID NO: 12 - protein, 5 aa, Synthetic construct
HYWMY

SEQ ID NO: 13 - protein, 17 aa, Synthetic construct
AINTGGGITYYTDSVKG

SEQ ID NO: 14 - protein, 17 aa, Synthetic construct
SRYGVVITTTYFGGVDY

SEQ ID NO: 15 - protein, 5 aa, Synthetic construct
NYGVH

SEQ ID NO: 16 - protein, 17 aa, Synthetic construct
HISAAGISRYYADAVKG

SEQ ID NO: 17 - protein, 11 aa, Synthetic construct
DRIRSSYEYDS

SEQ ID NO: 18 - protein, 5 aa, Synthetic construct
RSGMA

SEQ ID NO: 19 - protein, 17 aa, Synthetic construct
AITPGGTATYYANSVKG

SEQ ID NO: 20 - protein, 12 aa, Synthetic construct
GLVSSGLNGMDY

SEQ ID NO: 21 - protein, 5 aa, Synthetic construct
SYYMY

SEQ ID NO: 22 - protein, 17 aa, Synthetic construct
GTSIGGGSTWYADSVKG

SEQ ID NO: 23 - protein, 12 aa, Synthetic construct
DLNSRSYKGMGD

SEQ ID NO: 24 - protein, 14 aa, Synthetic construct
GLSSGSVTRNNYPD

SEQ ID NO: 25 - protein, 7 aa, Synthetic construct
NTVARHS

SEQ ID NO: 26 - protein, 13 aa, Synthetic construct
ALYMYTGSNNGRV

SEQ ID NO: 27 - protein, 11 aa, Synthetic construct
QGGNFGSYYAS

SEQ ID NO: 28 - protein, 7 aa, Synthetic construct
GNSNRPS

SEQ ID NO: 29 - protein, 10 aa, Synthetic construct
LSYESSGYAV

SEQ ID NO: 30 - protein, 14 aa, Synthetic construct
TGSSSNIGGGYYLS

SEQ ID NO: 31 - protein, 7 aa, Synthetic construct
NANNRAS

SEQ ID NO: 32 - protein, 12 aa, Synthetic construct
GCYDSSLSTGYV

SEQ ID NO: 33 - protein, 7 aa, Synthetic construct
NTNNRAS

SEQ ID NO: 34 - protein, 11 aa, Synthetic construct
GCYDDSLNTYV

SEQ ID NO: 35 - protein, 14 aa, Synthetic construct
TGSSSNIGGGYYLN

SEQ ID NO: 36 - protein, 11 aa, Synthetic construct
GCYDSSLSSVV

SEQ ID NO: 37 - protein, 121 aa, Synthetic construct
ELQLVESGGGLVQPGGSLRLSCAASGFTFDDYTMNWVRQAPGKGLEWVSAIRWNGVTTYYAESMKGRFTVSRDNGNNTLYLQMNSLKAEDTAVYYCAKGGSIDLTYGMDYWGKGTLVTVSS

SEQ ID NO: 38 - protein, 113 aa, Synthetic construct
QAVVTQEPSLSVSPGGTVTITCGLSSGSVTRNNYPDWYQQTPGQAPRLLLYNTVARHSGVPSRFSGSISGNKAALTITGAQPEDEAGYYCALYMYTGSNNGRVFGGGTLLTVL

SEQ ID NO: 39 - protein, 126 aa, Synthetic construct
QVQLVESGGGLVQPGGSLRLSCAASGFTFSHYWMYWVRQAPGKGLEWVSAINTGGGITYYTDSVKGRFTVSRDNAKNTLYLQMNSLKSEDTAVYYCAKSRYGVVITTTYFGGVDYWGKGTLVTVSS

SEQ ID NO: 40 - protein, 107 aa, Synthetic construct
SSALTQPSAVSVSLGQTARITCQGGNFGSYYASWYQQKPGQAPVQVIYGNSNRPSGIPERFSGSSSGDTATLTISGAQAEDEADYYCLSYESSGYAVFGGGTHLTVL

SEQ ID NO: 41 - protein, 120 aa, Synthetic construct
EVQLVESGGGLVQPGGSLRLSCAASGFTFSNYGVHWVRQAPGKGLEWVSHISAAGISRYYADAVKGRFTMSRDNGKNTLYLQMNGLKSEDTAVYYCAKDRIRSSYEYDSWGQGTQVTVSS

SEQ ID NO: 42 - protein, 112 aa, Synthetic construct
LPVLTQLSSMSGSPGQTVTITCTGSSSNIGGGYYLSWYQQLPGTAPKLLIYNANNRASGVPNRFSGSKSGNLASLTITGLQAEDEADYYCGCYDSSLSTGYVFGGGTKLTVL

SEQ ID NO: 43 - protein, 120 aa, Synthetic construct
LQLVESGGGLVQPGGSLRLSCAASGFTFSRSGMAWVRQAPGKGLEWVSAITPGGTATYYANSVKGRVTMSRDNAKNTLYLQMNSLKSEDTAVYYCAKGLVSSGLNGMDYWGKGTLVTVSS

SEQ ID NO: 44 - protein, 111 aa, Synthetic construct
QSVLTQLSSMSGSPGQTVTITCTGSSSNIGGGYYLSWYQQLPGTAPKLLIYNTNNRASGVPNRFSGSKTGSLASLTITGLQAEDEADYYCGCYDDSLNTYVFGGGTKLTVL

SEQ ID NO: 45 - protein, 121 aa, Synthetic construct
EVQLVESGGGLVQPGGSLRLSCAASGFTFSSYYMYWVRQAPGKGLEWVSGTSIGGGSTWYADSVKGRFTISRDNAKNTLYLQMNNLKSEDTAVYYCAKDLNSRSYKGMGDWGQGTQVTVSS

SEQ ID NO: 46 - protein, 111 aa, Synthetic construct
QPALTQLSSMSGSPGQTVTITCTGSSSNIGGGYYLNWYQQLPGAAPKLVIYNANNRASGVPDRFSGSKSGNLASLTITGLQAEDEAVYFCGCYDSSLSSVVFGGGTHLTVL

SEQ ID NO: 47 - protein, 5 aa, Synthetic construct
NYYMT

SEQ ID NO: 48 - protein, 17 aa, Synthetic construct
SIYSTGSHTYYADSVKG

SEQ ID NO: 49 - protein, 15 aa, Synthetic construct
NIYTYYTAHYAGMDY

SEQ ID NO: 50 - protein, 11 aa, Synthetic construct
QGGSLGSYDAH

SEQ ID NO: 51 - protein, 7 aa, Synthetic construct
DDNSRPS

SEQ ID NO: 52 - protein, 11 aa, Synthetic construct
HSYDSSANAPV

SEQ ID NO: 53 - protein, 124 aa, Synthetic construct
QVQLVESGGGLVQPGGSLTLSCAASGFTFSNYYMTWVRQAPGKGLEWVSSIYSTGSHTYYADSVKGRFTISRDNAKDTLYLQMNDLKSEDTAVYYCAKNIYTYYTAHYAGMDYWGKGTLVTVSS

SEQ ID NO: 54 - protein, 108 aa, Synthetic construct
QSALTQPSTVSVSLGQMARITCQGGSLGSYDAHWYQQKPGQAPVLVIHDDNSRPSGIPERFSGSRSGGTATLTISGAQADDEGVYFCHSYDSSANAPVFGGGTELTVL

SEQ ID NO: 55 - protein, 5 aa, Synthetic construct
SYWMD

SEQ ID NO: 56 - protein, 17 aa, Synthetic construct
RITNSGGTAFYTDSVEG

SEQ ID NO: 57 - protein, 13 aa, Synthetic construct
YITRAQLMGAWDY

SEQ ID NO: 58 - protein, 16 aa, Synthetic construct
KASQSLVHTDGKTYLS

SEQ ID NO: 59 - protein, 7 aa, Synthetic construct
QVSNRAS

SEQ ID NO: 60 - protein, 9 aa, Synthetic construct
AQSTYYPYD

SEQ ID NO: 61 - protein, 122 aa, Synthetic construct
EVQLVESGGDLVQPGGSLRLSCAASGFTFSSYWMDWVRQAPGKGLEWVSRITNSGGTAFYTDSVEGRFTISRDNAKNTLYLQMNSLKSEDTAVYYCLRYITRAQLMGAWDYWGQGTQVTVSS

SEQ ID NO: 62 - protein, 112 aa, Synthetic construct
ATMLTQSPGSLSVVPGESASISCKASQSLVHTDGKTYLSWLLQKPGQRPQLLIYQVSNRASGVPDRFTGSGSGTDFTLKISGVKAEDAGVYYCAQSTYYPYDFGSGTRLEIR

SEQ ID NO: 63-66 - skipped (4 entries)

SEQ ID NO: 67 - protein, 662 aa, Homo sapiens
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 68 - protein, 10 aa, Homo sapiens
EPKSCDKTHT

SEQ ID NO: 69 - protein, 5 aa, Homo sapiens
CPPCP

SEQ ID NO: 70 - protein, 8 aa, Homo sapiens
APELLGGP

SEQ ID NO: 71 - protein, 12 aa, Homo sapiens
ELKTPLGDTTHT

SEQ ID NO: 72 - protein, 50 aa, Homo sapiens
CPRCPEPKSCDTPPPCPRCPEPKSCDTPPPCPRCPEPKSCDTPPPCPRCP

SEQ ID NO: 73 - protein, 8 aa, Homo sapiens
APELLGGP

SEQ ID NO: 74 - protein, 7 aa, Homo sapiens
ESKYGPP

SEQ ID NO: 75 - protein, 5 aa, Homo sapiens
CPSCP

SEQ ID NO: 76 - protein, 8 aa, Homo sapiens
APEFLGGP

SEQ ID NO: 77 - skipped

SEQ ID NO: 78 - protein, 10 aa, Homo sapiens
CCVECPPPCP

SEQ ID NO: 79 - protein, 7 aa, Homo sapiens
APPVAGP

SEQ ID NO: 80 - protein, 22 aa, Homo sapiens
TGVVRQWLSRGGEIEGFRLSAH

SEQ ID NO: 81 - protein, 22 aa, Mus musculus
TGVVRQWLNQGDGIQGFRFSAH

SEQ ID NO: 82 - protein, 136 aa, Homo sapiens
RVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAH

SEQ ID NO: 83 - protein, 136 aa, Mus musculus
RVAGESADPEPEPEADYYAKEVTRVLMVDRNNAIYEKTKDISHSIYMFFNTSDLREAVPEPVLLSRAELRLQRLKSSVEQHVELYQKYSNNSWRYLGNRLLTPTDTPEWLSFDVTGVVRQWLNQGDGIQGFRFSAH

SEQ ID NO: 84 - protein, 49 aa, Homo sapiens
RVAGESAEPEPEPEADYYAKEVTRVLMVETHNEIYDKFKQSTHSIYMFF

SEQ ID NO: 85 - protein, 49 aa, Mus musculus
RVAGESADPEPEPEADYYAKEVTRVLMVDRNNAIYEKTKDISHSIYMFF

SEQ ID NO: 86 - protein, 65 aa, Homo sapiens
NTSELREAVPEPVLLSRAELRLLRLKLKVEQHVELYQKYSNNSWRYLSNRLLAPSDSPEWLSFDV

SEQ ID NO: 87 - protein, 65 aa, Mus musculus
NTSDLREAVPEPVLLSRAELRLQRLKSSVEQHVELYQKYSNNSWRYLGNRLLTPTDTPEWLSFDV